(19) United States
(12) Patent Application Publication
(10) Pub. No.: US 2025/0339156 A1
(43) Pub. Date: Nov. 6, 2025

(54) METHODS AND APPARATUS FOR IMPLANTING A FUSION DEVICE INTO A SACROILIAC JOINT

(71) Applicant: VGI Medical, LLC, Largo, FL (US)

(72) Inventor: Tov Inge Vestgaarden, Madeira Beach, FL (US)

(21) Appl. No.: 19/268,578

(22) Filed: Jul. 14, 2025

Related U.S. Application Data
(63) Continuation-in-part of application No. 18/515,470, filed on Nov. 21, 2023.
Application No. 18/515,470 is a continuation of application No. 15/889,587, filed on Feb. 6, 2018, now Pat. No. 11,819,226.
Application No. 15/889,587 is a continuation of application No. 13/790,416, filed on Mar. 8, 2013, now Pat. No. 9,883,874.
(63) Continuation-in-part of application No. 18/739,209, filed on Jun. 10, 2024.
Application No. 18/739,209 is a continuation-in-part of application No. 18/208,963, filed on Jun. 13, 2023.
Application No. 18/208,963 is a continuation of application No. 15/899,577, filed on Feb. 20, 2018, now Pat. No. 11,672,574.
Application No. 15/899,577 is a continuation of application No. 15/195,191, filed on Jun. 28, 2016, now Pat. No. 9,895,176.
Application No. 15/195,191 is a continuation of application No. 14/537,327, filed on Nov. 10, 2014, now Pat. No. 9,375,243.
Application No. 14/537,327 is a continuation of application No. 13/625,180, filed on Sep. 24, 2012, now Pat. No. 8,882,818.
(60) Provisional application No. 63/471,788, filed on Jun. 8, 2023.
(60) Provisional application No. 63/670,550, filed on Jul. 12, 2024.
(60) Provisional application No. 63/713,933, filed on Oct. 30, 2024.

(51) Int. Cl. A61B 17/17; A61B 17/70; A61B 17/88; A61B 17/90; A61F 2/46

(52) U.S. Cl. CPC A61B 17/1739; A61B 17/17; A61B 17/1757; A61B 17/8894; A61F 2/4603; A61F 2/4611; A61B 17/7055; A61B 17/90

(57) ABSTRACT

Apparatus for positioning a fusion implant into a sacroiliac joint, the apparatus comprising: a directional cannula comprising: a main body having a distal end, a proximal end and a lumen extending between the distal end and the proximal end; two cannula prongs extending distally from the distal end of the main body; a distal cut-out formed in the distal end of the main body; and a proximal notch formed in the proximal end of the main body so as to define a first proximal end surface and a second proximal end surface, wherein the first proximal end surface is disposed distal to the second proximal end surface; and a combined implant tamp and joint locator configured to be disposed in the lumen of the directional cannula, the combined implant tamp and joint locator comprising: a tamp body having a distal end and a proximal end; two tamp prongs extending distally from the distal end of the tamp body; and a proximal stop formed at the proximal end of the tamp body so as to define a first stop surface and a second stop surface, wherein the first stop surface is disposed distal to the second stop surface.

### REFERENCE TO PENDING PRIOR PATENT APPLICATIONS

This patent application:
(i) is a continuation-in-part of pending prior U.S. patent application Ser. No. 18/515,470, filed Nov. 21, 2023 by VGI Medical, LLC and Tov Inge Vestgaarden for TOOL AND METHOD FOR IMPLANTING FUSION DEVICE INTO SACROILIAC JOINT (Attorney's Docket No. VG-2425.10 CON 2), which patent application, in turn, is a continuation of prior U.S. patent application Ser. No. 15/889,587, filed Feb. 6, 2018 by VG Innovations, LLC and Tov Inge Vestgaarden for TOOL AND METHOD FOR IMPLANTING FUSION DEVICE INTO SACROILIAC JOINT (Attorney's Docket No. VG-2425.10 CON), which patent application, in turn, is a continuation of prior U.S. patent application Ser. No. 13/790,416, filed Mar. 8, 2013 by VG Innovations, LLC for TOOL AND METHOD FOR IMPLANTING FUSION DEVICE INTO SACROILIAC JOINT (Attorney's Docket No. VG-2425.10);
(ii) is a continuation-in-part of pending prior U.S. patent application Ser. No. 18/739,209, filed Jun. 10, 2024 by VGI Medical, LLC and Tov Inge Vestgaarden for APPARATUS AND METHOD FOR FUSING A SACROILIAC JOINT (Attorney's Docket No. VG-10), which patent application, in turn,
   (a) is a continuation-in-part of pending prior U.S. patent application Ser. No. 18/208,963, filed Jun. 13, 2023 by VGI Medical, LLC and Tov Inge Vestgaarden for METHOD FOR DEPLOYING A FUSION DEVICE FOR SACROILIAC JOINT FUSION (Attorney's Docket No. VG-2425.08 CON 4), which patent application, in turn, is a continuation of prior U.S. patent application Ser. No. 15/899,577, filed Feb. 20, 2018 by VGI Medical, LLC and Tov Inge Vestgaarden for METHOD FOR DEPLOYING A FUSION DEVICE FOR SACROILIAC JOINT FUSION (Attorney's Docket No. VG-2425.08. CON 3), which patent application, in turn, is a continuation of prior U.S. patent application Ser. No. 15/195,191, filed Jun. 28, 2016 by VGI Medical, LLC for METHOD FOR DEPLOYING A FUSION DEVICE FOR SACROILIAC JOINT FUSION (Attorney's Docket No. VG-2425.08. CON 2), which patent application, in turn, is a continuation of prior U.S. patent application Ser. No. 14/537,327, filed Nov. 10, 2014 by VG Innovations, LLC for METHOD FOR DEPLOYING A FUSION DEVICE FOR SACROILIAC JOINT FUSION (Attorney's Docket No. VG-2425.08. CON), which patent application, in turn, is a continuation of prior U.S. patent application Ser. No. 13/625,180, filed Sep. 24, 2012 by VG Innovations, LLC for METHOD FOR DEPLOYING A FUSION DEVICE FOR SACROILIAC JOINT FUSION (Attorney's Docket No. VG-2425.08); and
   (b) claims benefit of pending prior U.S. Provisional Patent Application Ser. No. 63/471,788, filed Jun. 8, 2023 by Tov Inge Vestgaarden and VGI Medical, LLC for ALTERNATIVE STABILIZATION IMPLANT (Attorney's Docket No. VG-10 PROV);
(iii) claims benefit of pending prior U.S. Provisional Patent Application Ser. No. 63/670,550, filed Jul. 12, 2024 by VGI Medical, LLC and Tov Inge Vestgaarden for IMPLANT DELIVERY SYSTEM COMPRISING NOVEL DIRECTIONAL CANNULA AND COMBINATION JOINT LOCATOR AND IMPLANT TAMP (Attorney's Docket No. VG-13 PROV); and
(iv) claims benefit of pending prior U.S. Provisional Patent Application Ser. No. 63/713,933, filed Oct. 30, 2024 by VGI Medical, LLC and Tov Inge Vestgaarden for METHOD AND APPARATUS FOR DELIVERING AN IMPLANT (Attorney's Docket No. VG-14 PROV).

The twelve (12) above-identified patent applications are hereby incorporated herein by reference.

### FIELD OF THE INVENTION

This invention relates to surgical instruments and methods in general, and more particularly to novel tools and methods for introducing an implant into a sacroiliac joint.

### BACKGROUND OF THE INVENTION

Lower back pain is a common ailment which often results in pain and suffering as well as loss of work time. Effective treatments for lower back pain will alleviate considerable patient suffering and provide economic benefits by reducing employee absenteeism.

In the past, lower back pain and leg pain have been attributed to herniated discs or other injuries to the spinal column. However, it has been recognized that extensive therapy and treatment is often unsuccessful in alleviating such pain. More recently, it has been recognized that at least some forms of lower back and leg pain can be attributed to symptomatic sacroiliac joint dysfunction or instability of the sacroiliac joint.

The sacroiliac joint is located at the juncture of the ilium (i.e., the upper bone of the pelvis, commonly called the hip bone), and the sacrum (i.e., located at the base of the lumbar spine, where it connects with the L5 vertebra). The function of the sacroiliac joint is the transmission of forces from the spine to the lower extremities and vice-versa. The sacroiliac joint is supported by a range of ligaments, including the sacroiliac ligament at the base of the joint and the anterior sacroiliac ligament at the top of the joint.

The sacroiliac joint has a limited range of motion. Nutation, the relative movement between the sacrum and ilium, is typically only about one to two degrees. Despite the limited range of motion between the sacrum and the ilium, a patient's sacroiliac joint can become damaged resulting in hypermobility of the joint. Hypermobility of the sacroiliac joint is very difficult to diagnose due to the small range of motion. Therefore, lower back pain or leg pain caused by sacroiliac joint dysfunction (e.g., degenerative sacroiliitis, inflammatory sacroiliitis, iatrogenic instability of the sacroiliac joint, osteitis condensans ilii, traumatic fracture dislocation of the pelvis, etc.) often goes misdiagnosed or undiagnosed.

In patients where sacroiliac joint pain is unresponsive to non-operative treatments, e.g. medication, physical therapy, chiropractic care and steroid injections, surgical stabilization is prescribed. Joint fusion is a surgical treatment that may be employed in order to relieve pain generated from sacroiliac joint dysfunction.

Placing an implant (sometimes also referred to herein as a “fusion implant”) into the sacroiliac joint space is challenging because the ilium protrudes and blocks easy access to the sacroiliac joint. Part of the ilium can be cut and removed in order to improve access, however, such cutting weakens the ilium, extends the time required for surgery and recovery from surgery, and increases the patient's pain.

In addition, there can exist a significant amount of soft tissue between the patient's skin and the sacroiliac joint. Surgeons generally wish to minimize soft tissue trauma to the patient, even when accessing a joint that is a significant distance away from the patient's skin. Since fusion of the sacroiliac joint generally requires delivering an implant into the sacroiliac joint so as to span the joint space, and since such implants tend to be extremely small while also requiring delivery into the joint in a particular prescribed orientation, significant retraction of tissue is generally needed in order to fully expose the sacroiliac joint.

Ideally such a joint fusion procedure can be performed in a minimally-invasive manner by utilizing a cannula to deliver the implant (and such other surgical tools as are required for the procedure) to the joint space. However, the aforementioned protruding ilium creates an obstacle that must be accommodated by such a cannula. Furthermore, it is also desirable to reduce the total number of surgical tools that must be employed for such a procedure in order to reduce the length and/or cost of the procedure.

Thus there is a need for novel method and apparatus for delivering an implant to the sacroiliac joint which accommodates the surrounding anatomy and reduces the total number of surgical tools needed for the joint fusion procedure, whereby to allow for correcting symptomatic sacroiliac joint dysfunction or instability, enhancing stability for purposes of immobilizing a joint, and fusing two opposed bone structures across the joint.

### SUMMARY OF THE INVENTION

The present invention comprises novel methods and apparatus for delivering an implant to the sacroiliac joint which accommodates the surrounding anatomy and reduces the total number of surgical tools needed for the joint fusion procedure, whereby to allow for correcting symptomatic sacroiliac joint dysfunction or instability, enhancing stability for purposes of immobilizing a joint, and fusing two opposed bone structures across the joint.

Specifically, the novel apparatus of the present invention includes a directional cannula having a main body of elongate cylindrical extent. A bore having the transverse profile of an implant to be passed through the bore is formed in the main body of the directional cannula, which bore is also configured to receive a drill guide therein (if desired).

A cut-out is formed in a leading end of the main body by a radial cut, i.e., a cut that is generally normal to a longitudinal axis of the main body of the directional cannula. The cut-out allows the directional cannula to be used in the region of the protruding ilium, as the cut-out provides a region to accommodate a portion of the ilium, thereby eliminating the need to cut the ilium itself. The radial cut extends less than half-way through the main body of the directional cannula. The cut-out is formed by a second longitudinally-extending cut that extends from a leading end of the main body of the directional cannula to the radial cut at a point of deepest penetration of the radial cut.

The implant of the present invention is captured within the bore of the directional cannula while the implant is passed through the cut-out (or notch) formed in the main body of the directional cannula and is exposed to view. Since the notch has a radial depth less than the diameter of the bore, the implant is more than half-surrounded by the lumen and is therefore slidably held within the bore of the directional cannula.

A pair of parallel prongs preferably extend longitudinally from the leading end of the main body of the directional cannula in transversely spaced apart relation to one another. The prongs are adapted to enter the sacroiliac joint so as to locate and distract the sacroiliac joint.

In a preferred embodiment of the invention, there is also provided a novel drill guide having an elongate main body, an enlarged cylindrical head formed integrally with the main body at a proximal end of the main body, and a transverse width-reducing step formed in the main body near a distal end of the main body.

A first longitudinally-extending bore is formed in the enlarged cylindrical head and in the main body of the drill guide. The first bore is disposed eccentric relative to a longitudinal axis of symmetry of the drill guide.

A second longitudinally-extending bore is also formed in the enlarged cylindrical head and in the main body of the drill guide. The second bore is also disposed eccentric relative to the longitudinal axis of symmetry of the drill guide and the second bore extends parallel to the first bore.

A drill bit is sequentially placed within the first and second bores of the drill guide while the drill guide is disposed in the bore of the directional cannula, whereby to create clearance space by drilling in a portion of the ilium located in the region of the sacroiliac joint. The drill guide is then removed from the bore of the directional cannula, rotated one hundred eighty degrees (180°), and the drill bit is again sequentially placed within the first and second bores of the drill guide while the drill guide is disposed in the bore of the directional cannula, whereby to create clearance space by drilling in a portion of the sacrum located in the region of the sacroiliac joint.

In another preferred embodiment of the invention there is provided a novel implant delivery system comprising a directional cannula and a combined joint locator/implant tamp which eliminates the need for a separate joint locator and a separate implant tamp.

In another preferred form of the present invention, there is provided apparatus for positioning a fusion implant into a sacroiliac joint, the apparatus comprising:
a directional cannula comprising:
   a main body having a distal end, a proximal end and a lumen extending between the distal end and the proximal end;
   two cannula prongs extending distally from the distal end of the main body;
   a distal cut-out formed in the distal end of the main body; and
   a proximal notch formed in the proximal end of the main body so as to define a first proximal end surface and a second proximal end surface, wherein the first proximal end surface is disposed distal to the second proximal end surface; and
a combined implant tamp and joint locator configured to be disposed in the lumen of the directional cannula, the combined implant tamp and joint locator comprising:
   a tamp body having a distal end and a proximal end;
   two tamp prongs extending distally from the distal end of the tamp body; and
   a proximal stop formed at the proximal end of the tamp body so as to define a first stop surface and a second stop surface, wherein the first stop surface is disposed distal to the second stop surface.

In another preferred form of the present invention, there is provided a method of positioning a fusion implant into a sacroiliac joint, the method comprising:
inserting a combined implant tamp and joint locator into a lumen of a directional cannula;
inserting the directional cannula and the combined implant tamp and joint locator into the sacroiliac joint to prepare a cavity in the sacroiliac joint for receiving the fusion implant;
withdrawing the combined implant tamp and joint locator from the lumen of the directional cannula;
inserting the fusion implant into the lumen of the directional cannula;
re-inserting the combined implant tamp and joint locator into the lumen of the directional cannula; and
using the combined implant tamp and joint locator to tamp the fusion implant into the cavity in the sacroiliac joint.

In another preferred form of the present invention, there is provided a method of positioning a fusion implant into a sacroiliac joint, the method comprising:
providing a directional cannula comprising:
   a main body having a distal end, a proximal end and a lumen extending between the distal end and the proximal end;
   two cannula prongs extending distally from the distal end of the main body;
   a distal cut-out formed in the distal end of the main body; and
   a proximal notch formed in the proximal end of the main body so as to define a first proximal end surface and a second proximal end surface, wherein the first proximal end surface is disposed distal to the second proximal end surface;
providing a combined implant tamp/joint locator comprising:
   a tamp body having a distal end and a proximal end;
   two tamp prongs extending distally from the distal end of the tamp body; and
   a proximal stop formed at the proximal end of the tamp body so as to define a first stop surface and a second stop surface, wherein the first stop surface is disposed distal to the second stop surface;
inserting the combined implant tamp and joint locator into the lumen of the directional cannula in a first radial position such that the first stop surface of the combined implant tamp and joint locator engages the first proximal end surface of the directional cannula and distalmost ends of the two cannula prongs of the directional cannula are aligned with distalmost ends of the two tamp prongs of the combined implant tamp and joint locator;
inserting the directional cannula and the combined implant tamp and joint locator into the sacroiliac joint such that the two cannula prongs and the two tamp prongs engage the sacroiliac joint;
withdrawing the combined implant tamp and joint locator from the lumen of the directional cannula;
inserting the fusion implant into the lumen of the directional cannula;
rotating the combined implant tamp and joint locator 180 degrees to a second radial position;
inserting the rotated combined implant tamp and joint locator into the lumen of the directional cannula in the second radial position;
applying a force to the proximal end of the combined implant tamp and joint locator to seat the fusion implant in the sacroiliac joint, wherein the second stop surface of the combined implant tamp and joint locator and the second proximal end surface of the directional cannula prevent the two tamp prongs of the combined implant tamp and joint locator from extending distal to the two cannula prongs of the directional cannula; and
removing the combined implant tamp and joint locator and directional cannula from the sacroiliac joint.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1A is an X-ray view depicting the first guide wire placed on the skin (i.e., external to the sacroiliac joint) to identify the location of the sacroiliac joint;

FIG. 1B is a diagrammatic view of a mark made on a patient's skin to indicate the position of the guide wire used to locate the sacroiliac joint in FIG. 1A;

FIG. 2A is an X-ray view depicting the second guide wire placed on the skin (i.e., external to the sacroiliac joint) to identify the superior portion of the sacroiliac joint;

FIG. 2B is a diagrammatic view of a mark made on a patient's skin to indicate the position of the guide wire used to identify the superior portion of the sacroiliac joint in FIG. 2A;

FIG. 3A is an X-ray view depicting the third guide wire placed on the skin (i.e., external to the sacroiliac joint) to identify the inferior portion of the sacroiliac joint;

FIG. 3B is a diagrammatic view of a mark made on a patient's skin to indicate the position of the guide wire used to identify the inferior portion of the sacroiliac joint in FIG. 3A;

FIG. 4 is a diagrammatic view of three incision points that are marked on the patient's skin after the markings in FIGS. 1B, 2B, and 3B have been made;

FIG. 5 is an X-ray view depicting a guide wire inserted through the central incision point of the three incision points of FIG. 4;

FIG. 6A is a perspective view of a joint locator;

FIG. 6B is a side elevational view of the joint locator;

FIG. 6C is a top plan view of the joint locator;

FIG. 7A is a perspective view of a novel directional cannula;

FIG. 7B is a top plan view of the directional cannula of FIG. 7A;

FIG. 7C is a side elevational view of the directional cannula of FIG. 7A;

FIG. 7D is a bottom plan view of the directional cannula of FIG. 7A;

FIG. 7E is an end view of the leading end of the directional cannula of FIG. 7A;

FIG. 7F is an end view of the trailing end of the directional cannula of FIG. 7A;

FIG. 7G is a side elevational view similar to FIG. 7C, but depicting an implant exposed to view by a notch but captured within a lumen so that it cannot fall;

FIG. 7H is an end view taken along lines 7H-7H in FIG. 7G;

FIG. 8A is a perspective view of a novel drill guide;

FIG. 8B is a side elevational view of the drill guide of FIG. 8A;

FIG. 8C is an end elevational view of the leading end of the drill guide of FIG. 8A;

FIG. 8D is an end elevational view of the trailing end of the drill guide of FIG. 8A;

FIG. 9 is a perspective view of a drill bit;

FIG. 10 is a perspective view of a fusion implant device;

FIG. 11 is a perspective view of a tamp;

FIG. 12A is a front elevational diagrammatic representation of a sacrum flanked by a pair of iliums, showing two (2) sacroiliac joints;

FIG. 12B is a first perspective view of the diagrammatic representation of FIG. 12A, indicating how the iliums protrude forwardly relative to the sacrum;

FIG. 12C is a second perspective view of the diagrammatic representation of FIG. 12A;

FIG. 12D is a view like FIG. 12C but adding a notched cannula, showing how the notch accommodates a protruding ilium;

FIG. 12E depicts the same parts as FIG. 12D but taken from a left perspective;

FIG. 12F is the same view as FIG. 12E but including the drill bit;

FIG. 12G is the same as FIG. 12F but taken from a right perspective;

FIG. 12H depicts the cavity after drilling is complete and the cannula and drill bit have been withdrawn;

FIG. 12I is a like FIG. 12H but from a different perspective;

FIG. 12J depicts an implant in the drilled cavity;

FIG. 12K depicts the same structure as FIG. 12J but from a different perspective;

FIGS. 13-18 are schematic views showing an alternative novel implant delivery system formed in accordance with the present invention; and

FIGS. 19-32 are schematic views showing a method for using a novel implant delivery system in accordance with the present invention.

### DETAILED DESCRIPTION OF THE PREFERRED EMBODIMENTS

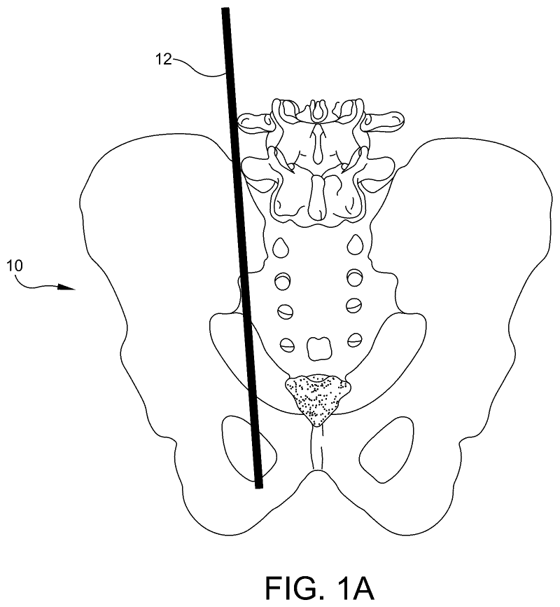

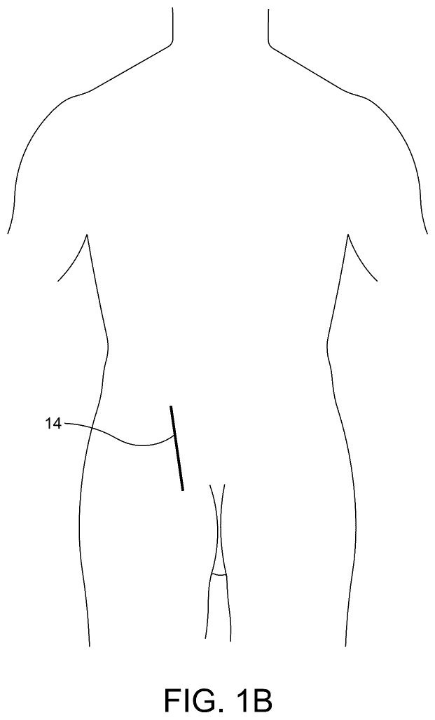

FIGS. 1A-8D depict an illustrative embodiment of a first embodiment of a novel implant delivery system and method for effecting fusion of a sacroiliac joint 10.

The novel method of the present invention comprises the steps of taking anterior, posterior, and lateral X-ray views of the sacroiliac (SI) area to identify the anatomy that is causing pain in a patient.

An entry point is established to gain access to the SI joint with an oblique (approximately thirty five degree (35°) angle) and a Ferguson angle measurement of curvature which is approximately ten to fifteen degrees (10-15°).

Looking first at FIG. 1A, while using an imaging device (e.g., X-ray, fluoroscopy) to image the SI joint, a first guide wire 12 is placed on top of the skin over the SI joint. Then, as depicted in FIG. 1B, the patient's skin is marked with a first straight line 14 to indicate the position of first guide wire 12 over the SI joint. First straight line 14 is drawn through SI joint 10 from the superior position of the joint to the inferior position of the joint.

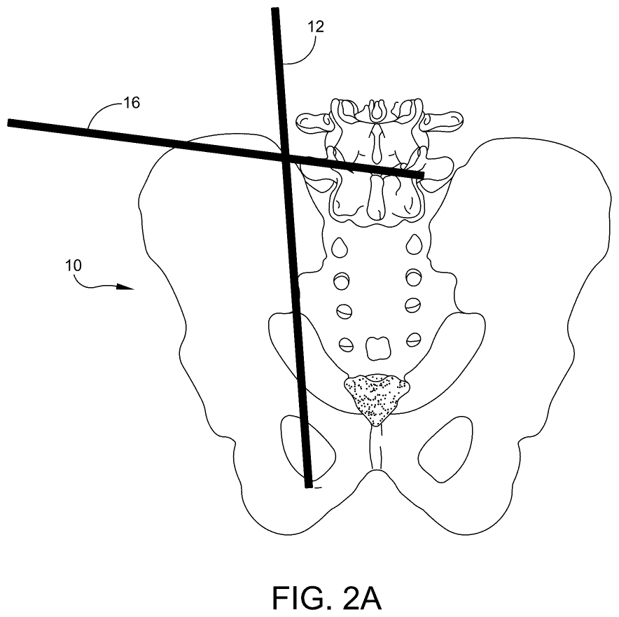

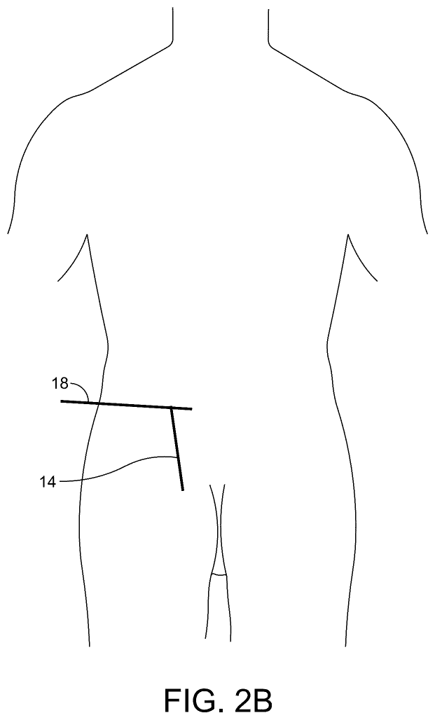

Looking next at FIG. 2A, while using an imaging device (e.g., X-ray, fluoroscopy) to image the SI joint, a second guide wire 16 is placed on top of the skin over the superior section of joint 10 in intersecting relation to first guide wire 12. Then, as depicted in FIG. 2B, the patient's skin is marked with a second straight line 18 to indicate the position of second guide wire 16.

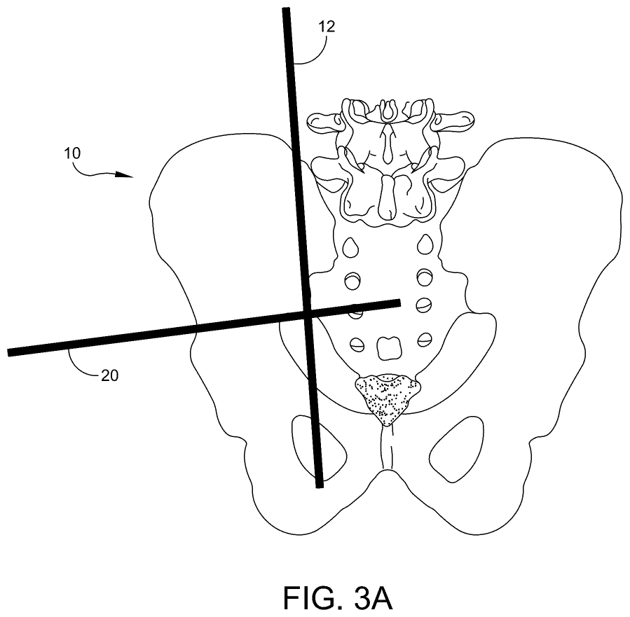

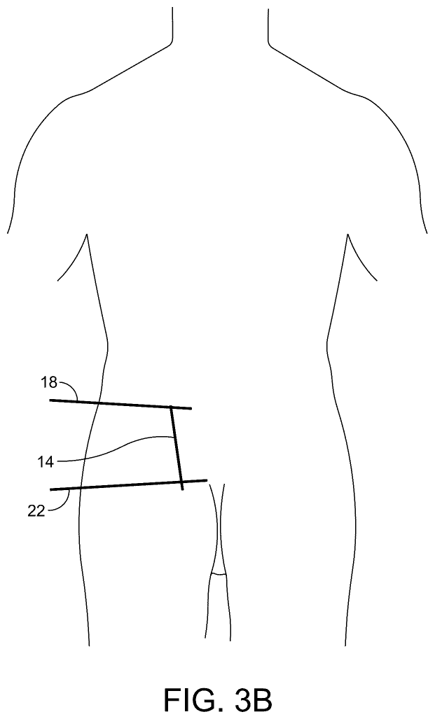

Looking next at FIG. 3A, while using an imaging device (e.g., X-ray, fluoroscopy) to image the SI joint, a third guide wire 20 is placed on top of the skin over the inferior section of joint 10 in intersecting relation to first guide wire 12. Then, as depicted in FIG. 3B, the patient's skin is marked with a third straight line 22 to indicate the position of third guide wire 20.

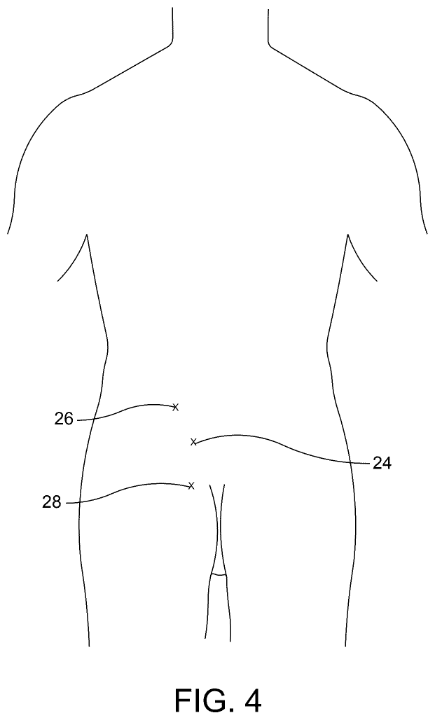

Three incision points 24, 26 and 28 are then marked on the skin, as depicted in FIG. 4.

Center incision point 24 is midway between the points where the second and third lines 18 and 22 cross first line 14 and is spaced in the medial direction about three to five millimeters (3-5 mm) from first line 14.

Superior incision point 26 is spaced in the medial direction about three to five millimeters (3-5 mm) from the intersection of first line 14 and second line 18.

Inferior incision point 28 is spaced in the medial direction about three to five millimeters (3-5 mm) from the intersection of first line 14 and third line 22.

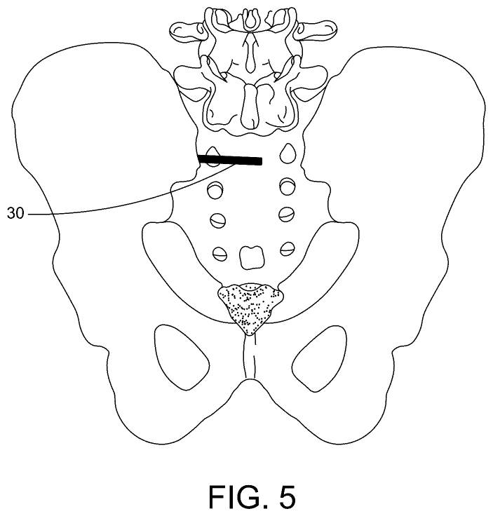

As depicted in FIG. 5, a fourth guide wire 30 is inserted through center incision point 24, with a superior/inferior angle perpendicular to the patient. The medial/lateral angle is the same as the oblique angle on the C-arm, which is approximately thirty-five degrees (35°). Fourth guide wire 30 is guided into the SI joint and an incision is made when guide wire 30 is properly positioned.

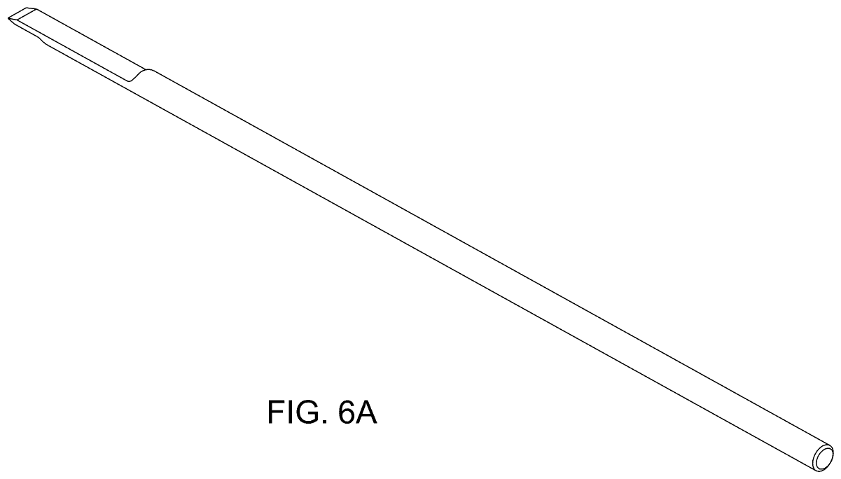

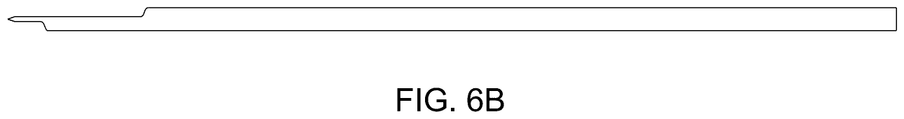

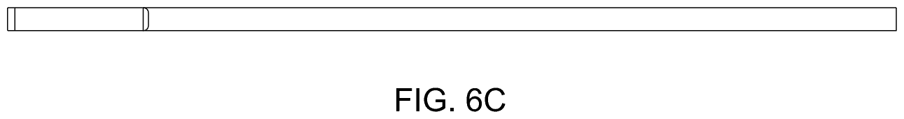

Joint locator 50, depicted in FIGS. 6A-C, is then placed over guide wire 30, i.e., in ensleeving relation to said guide wire 30. A surface of joint locater 50 is etched black and that black-etched surface is positioned so that it faces the ilium.

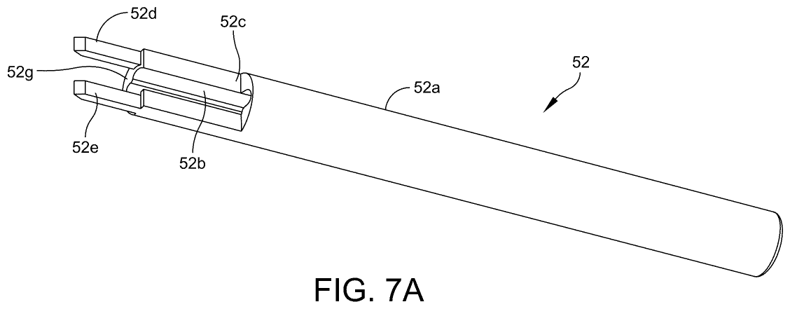

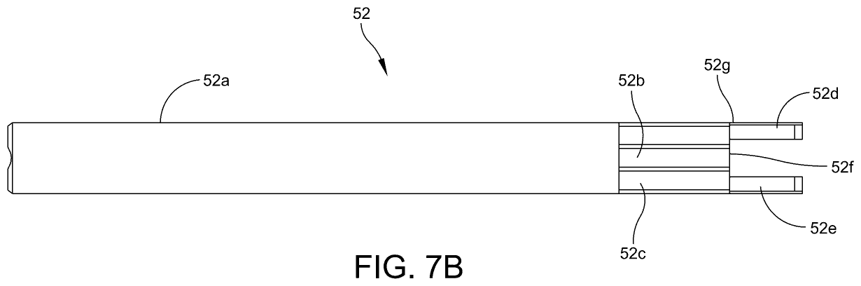

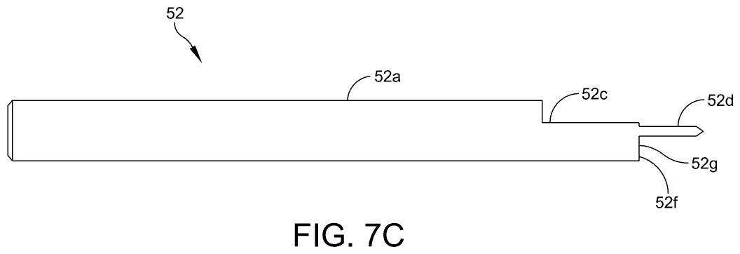

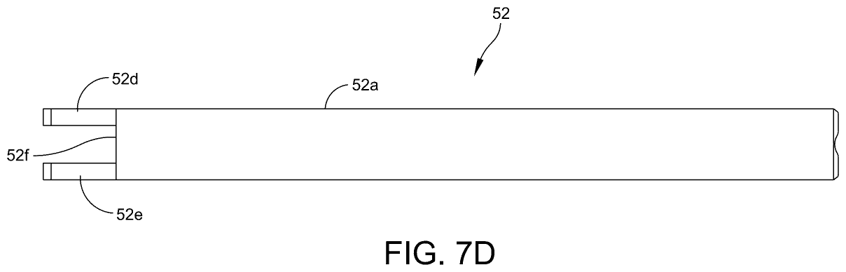

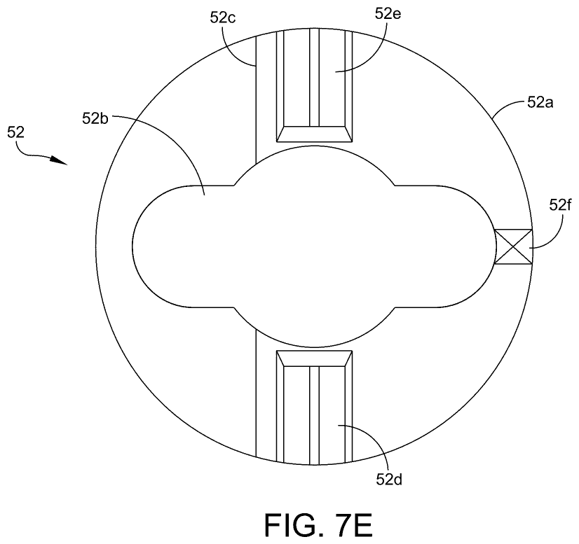

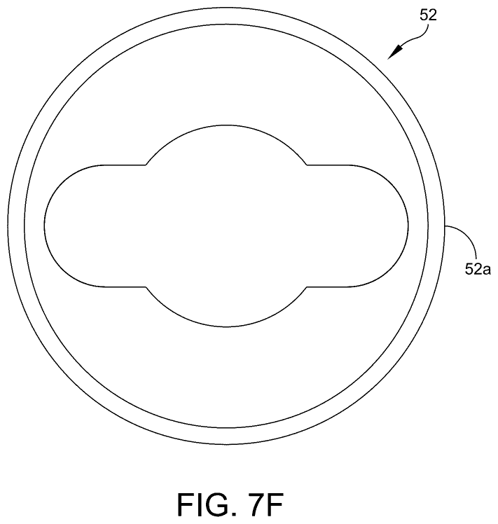

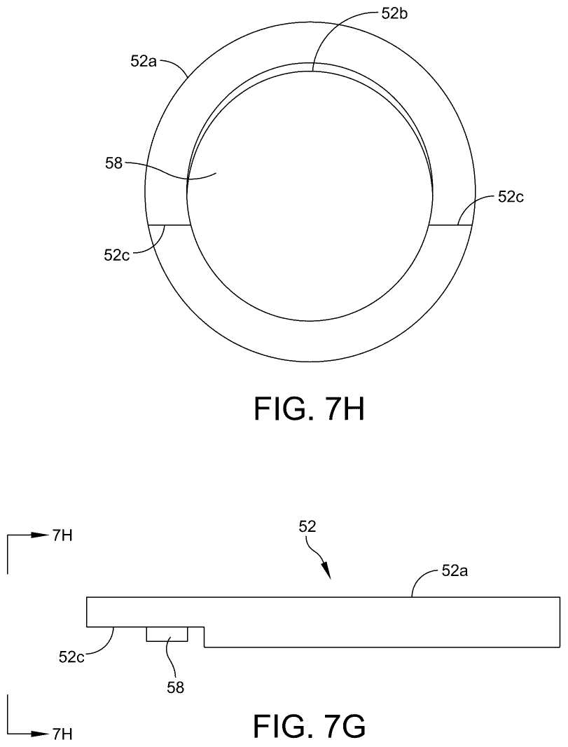

Directional cannula 52, depicted in FIGS. 7A-7G, is then placed over joint locator 50, i.e., in ensleeving relation to said joint locator. A surface of directional cannula 52 is etched black and that black-etched surface matches the black-etched surface of joint locator 50, i.e., the black-etched surface of directional cannula 52 is also positioned so that it faces the ilium.

Directional cannula 52 has a cylindrical main body 52a having longitudinally-extending central bore 52b formed therein. Central bore 52b has a transverse profile that matches the transverse profile of a fusion implant. A leading end 52g of central bore 52b is exposed to view by cut-out or notch 52c. Two prongs 52d and 52e extend from said leading end in transversely spaced, parallel relation to one another. Prongs 52d and 52e enter the sacroiliac joint (i.e., the space between the sacrum and the ilium) when directional cannula 52 is in use. Barb 52f is formed in the leading end of main body 52a as depicted in FIGS. 7B-7E. Barb 52f engages the sacrum to prevent slippage when prongs 52d and 52e enter into sacroiliac joint 10.

FIG. 7G depicts implant 58 that is captured within bore 52b. Implant 58 is exposed to view because it is in the region of bore 52b where notch 52c is formed. Since the radial depth of notch 52c is less than half the diameter of bore 52b, implant 58 cannot fall from bore/lumen 52b. In other words, as indicated in FIG. 7H, notch 52c creates a “C”-shaped bore where the two (2) spaced apart points of the “C” are closer together than the widest part of implant 58, thereby retaining the implant within bore 52b.

Joint locator 50 and guide wire 30 are then retracted, leaving directional cannula 52 in position. When so positioned, prongs 52d, 52e are disposed in sacroiliac joint 10. Barb 52f engages the sacrum to hold directional cannula 52 in position as aforesaid with notch 52c accommodating the ilium.

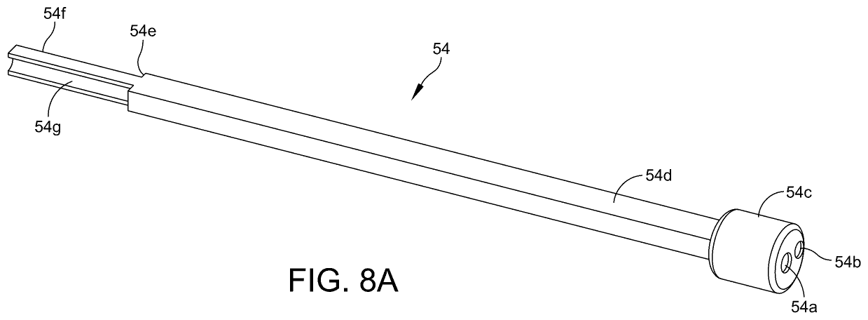

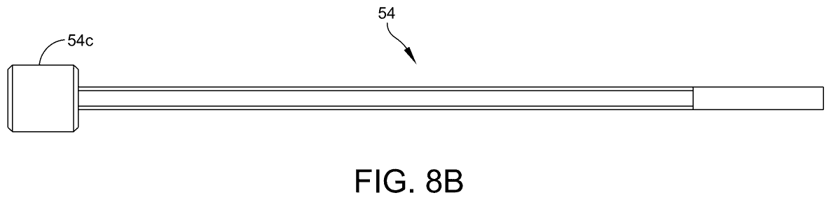

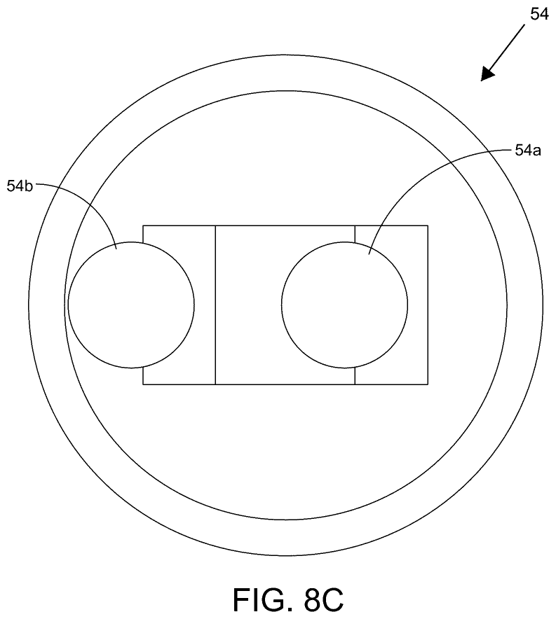

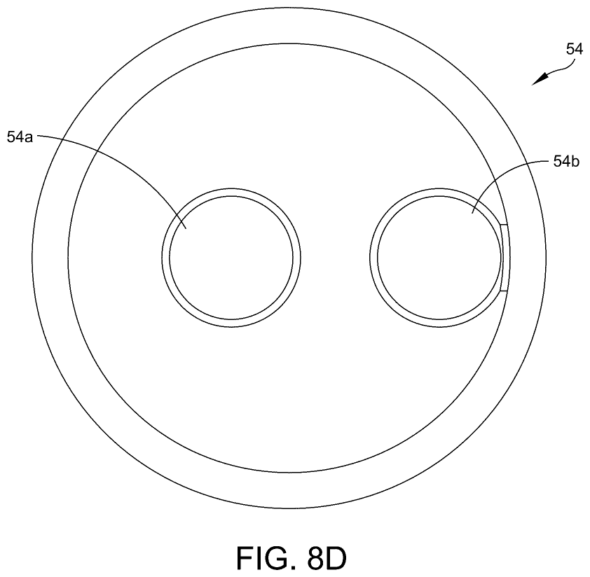

Drill guide 54, depicted in FIGS. 8A-8D, has longitudinally extending eccentric bores 54a, 54b formed therein. Head 54c is enlarged relative to main body 54d that is rectangular in transverse section. Step 54e is formed where the transverse extent of main body 54d is reduced. Drill bit-accommodating semi-circular grooves 54f and 54g are the continuation of bores 54a, 54b and are formed in opposite sides of the leading end of drill guide 54, said leading end being the part of main body 54d that extends distal of said step 54e.

Drill guide 54 is inserted into the central bore or lumen of directional cannula 52 towards the sacrum to verify placement of directional cannula 52 into the SI joint. The lumen of directional cannula 52 has a profile that enables it to slidingly receive drill guide 54 in the center of the larger implant-receiving lumen.

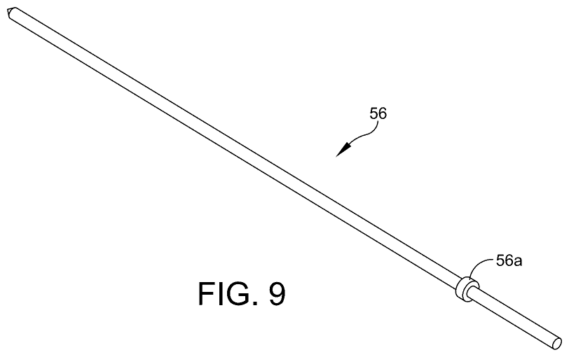

Drill bit 56, depicted in FIG. 9, having positive stop 56a is then inserted into eccentric bore 54a and said drill bit 56 is slid towards the sacrum until it abuts the sacrum.

The distance from the proximal end of head 54c of drill guide 54 to the lower side of positive stop 56a is then measured. Directional cannula 52 is properly seated in the SI joint if the measured distance is between twenty-five to thirty millimeters (25-30 mm). The proper seating can also be confirmed with a lateral X-ray view that shows the leading end of directional cannula 52 disposed flush with the sacrum.

Drill bit 56 is then inserted into eccentric drill guide bore 54a and a first cavity is created in the ilium by a first drilling, until positive stop 56a abuts the proximal end of directional cannula 52. Drill bit 56 is then withdrawn from bore 54a, placed into bore 54b, and a second cavity is formed in the ilium by a second drilling. The second drilling continues until drill bit 56 reaches positive stop 56a.

Drill guide 54 is then retracted from directional cannula 52 and rotated one hundred eighty degrees (180°). Drill bit 56 is then inserted into eccentric drill guide bore 54a and a first cavity is created in the sacrum by a third drilling that continues until drill bit 56 reaches positive stop 56a. Drill bit 56 is then withdrawn from bore 54a, placed into bore 54b, and a second cavity is formed in the sacrum by a fourth drilling. The fourth drilling continues until drill bit 56 reaches positive stop 56a. Drill bit 56 is then removed.

Due to the eccentricity of the bores and the rotation of the drill guide, all four cavities merge into a single cavity that accommodates the fusion implant.

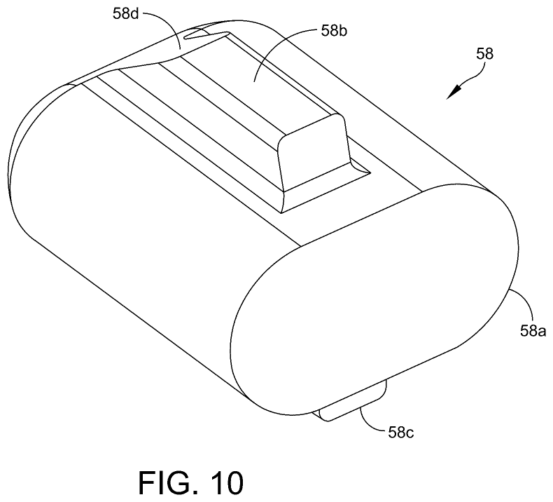

When the drilling is completed, drill guide 54 is retracted from directional cannula 52 and fusion implant 58, depicted in FIG. 10, is inserted into the lumen of directional cannula 52. The chamfer is inserted downward.

Fusion implant 58 may take many forms and may be as simple as a dowel having a circular cross-section, i.e., the oval shape of main body 58a, upper and lower fins 58b, 58c, and the swept back leading edge 58d of said fins are not critical parts of the fusion implant.

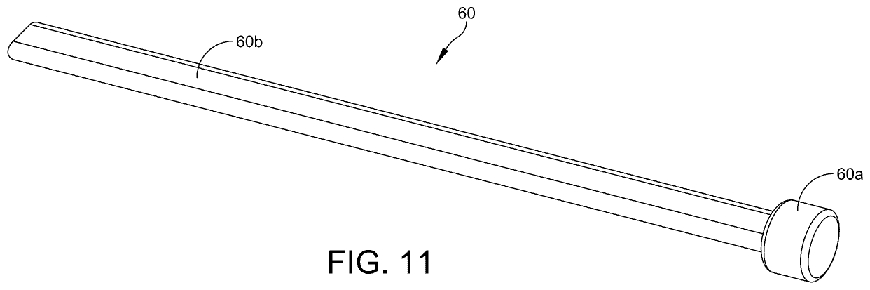

Implant tamp 60, depicted in FIG. 11, includes head 60a and flat, elongate main body 60b. A positive stop, not numbered, is formed where main body 60b meets head 60a. Main body 60b is inserted into the lumen of directional cannula 52 to advance the implant. Head 60a is repeatedly tamped lightly with a hammer or other suitable tool, not illustrated, until the aforesaid positive stop abuts directional cannula 52. This should fully seat implant 58 in the SI joint, i.e., implant 58 should be countersunk into the SI joint by a distance of about three to five millimeters (3-5 mm). A lateral X-ray view is taken to confirm full deployment of implant 58.

As perhaps best understood in connection with FIGS. 7B and 7C, implant 58 emerges from bore 52b at cut-out or notch 52c. Notch 52c has an extent or depth less than half the diameter of cylindrical main body 52a as depicted in FIGS. 7C and 7E. Implant 58 is thus supported from below by the part of main body 52a that is not removed to form notch 52c. The dimensions of implant 58 allow it to be inserted into bore 52b, but prevent it from falling out upon exiting bore 52b, i.e., upon exposure to or open communication with notch 52c, i.e., implant 58 is supported from below by the longitudinally-extending part of notch 52c as perhaps best understood in connection with FIG. 7C. Implant 58 is inside the cavity drilled into the sacrum and ilium when said implant extends beyond leading end 52g of cylindrical main body 52a.

The following procedure is then followed to insert additional implants 58 into cavities formed in the sacrum and the ilium.

A guide wire is inserted through superior incision point 26 at a superior/interior angle about forty-five degrees (45°) towards superior. The medial/lateral angle is the same as the oblique angle on the C-arm, which is approximately thirty-five degrees (35°).

The guide wire is then guided into the SI joint. A second incision is made when the guide wire is properly positioned. The steps that follow the first incision are then repeated, i.e., joint locator 50 is inserted over the guide wire, directional cannula 52 is paced over the joint locator, and so on.

The procedure is concluded by inserting a guide wire through inferior incision point 28 at a superior/interior angle about forty-five degrees (45°) towards inferior. The medial/lateral angle is the same as the oblique angle on the C-arm, which is approximately thirty-five degrees (35°).

The guide wire is then guided into the SI joint. A third incision is made when the guide wire is properly positioned. The steps that follow the first and second incisions are then repeated, i.e., joint locator 50 is inserted over the guide wire, directional cannula 52 is paced over the joint locator, and so on.

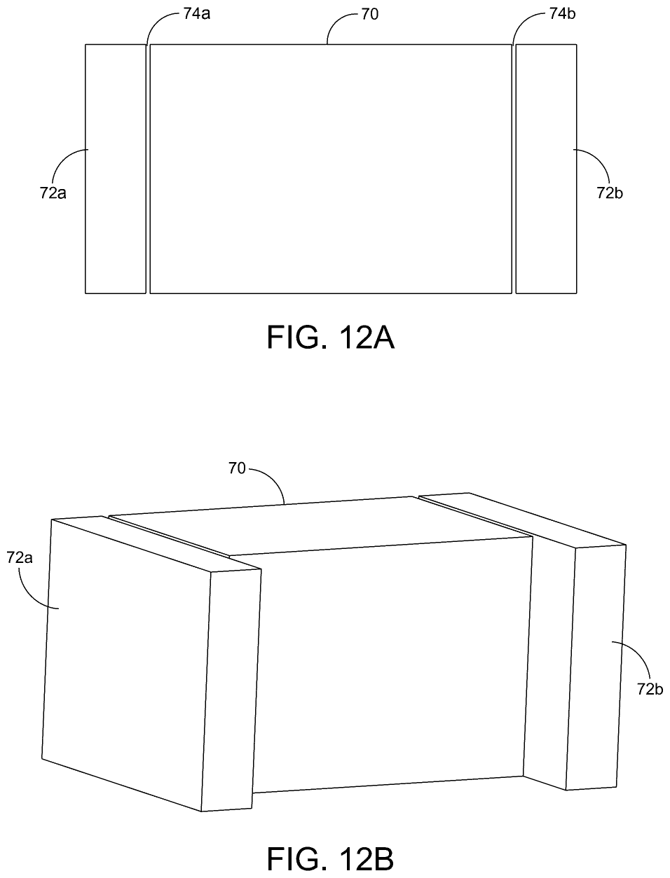

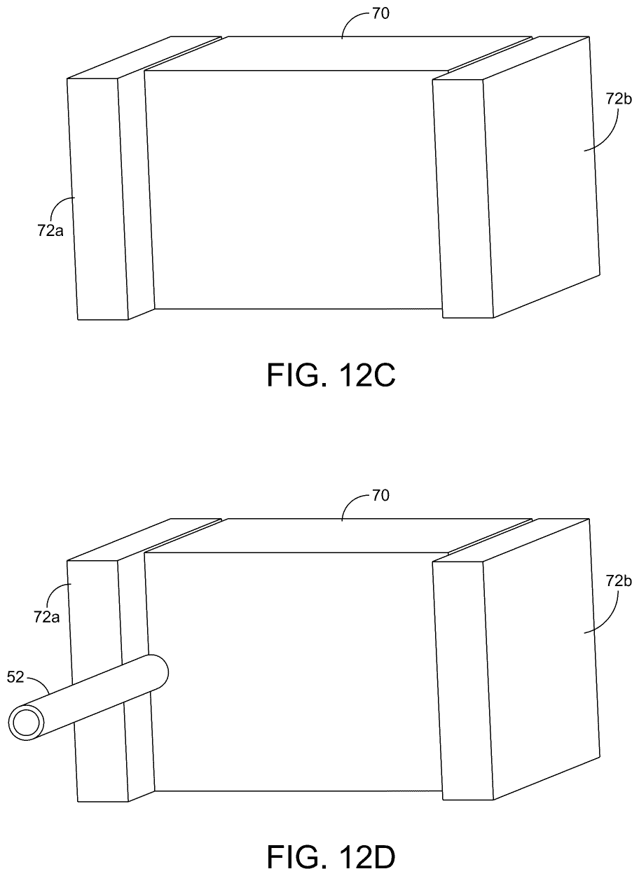

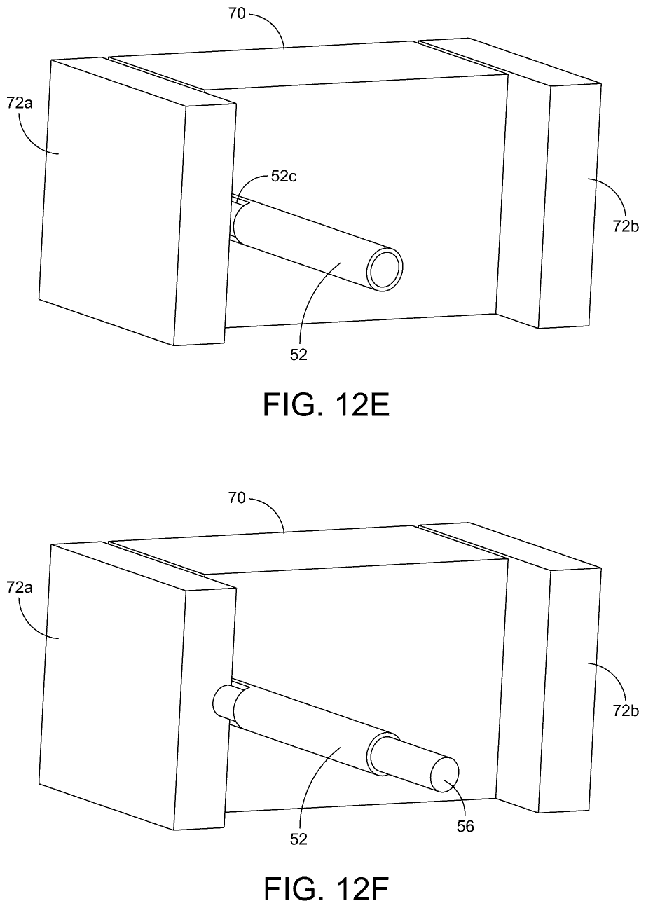

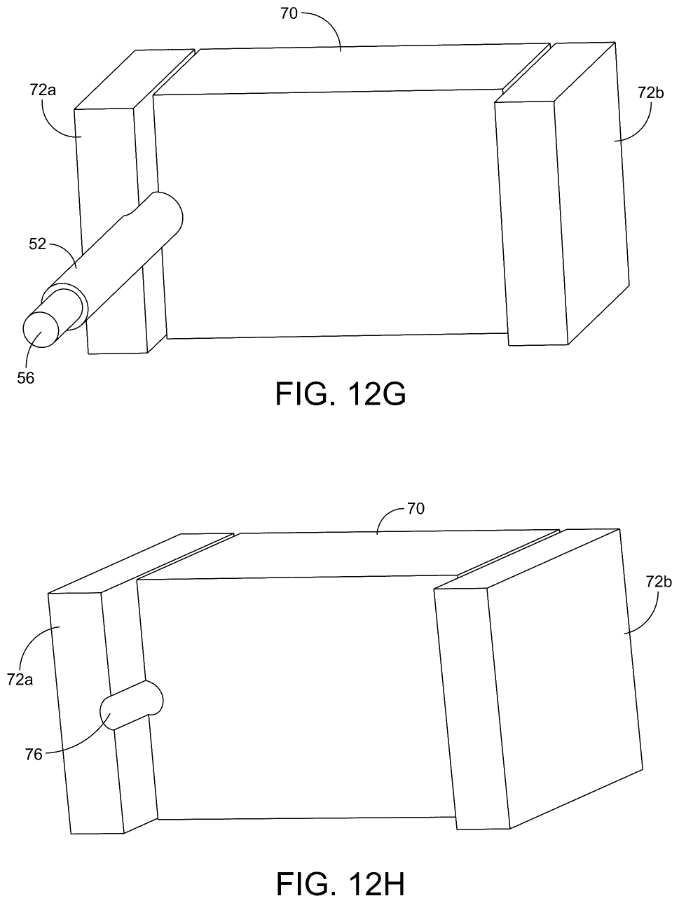

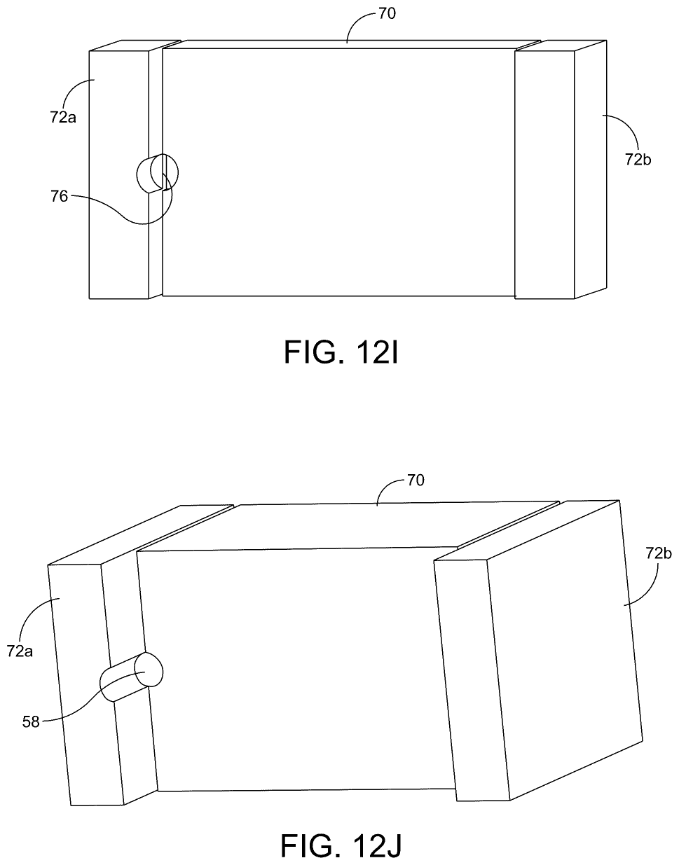

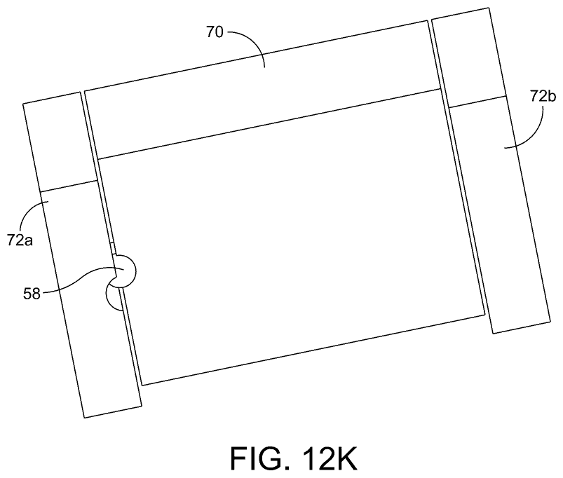

FIGS. 12A-K provide a simplified overview of the procedure. FIG. 12A is a front elevational diagrammatic representation of a sacrum 70 flanked by a pair of iliums 72a, 72b, showing two (2) sacroiliac joints 74a, 74b and FIG. 12B is a first perspective view of the diagrammatic representation of FIG. 12A, indicating how iliums 72a, 72b protrude forwardly relative to sacrum 70. FIG. 12C is a second perspective view of the diagrammatic representation of FIG. 12A and FIG. 12D is a view like FIG. 12C but adding notched cannula 52, showing how notch 52c accommodates a protruding ilium. FIG. 12E depicts the same parts as FIG. 12D but taken from a left perspective and FIG. 12F is the same view as FIG. 12E but including drill bit 56. FIG. 12G is the same as FIG. 12F but taken from a right perspective. FIG. 12H depicts cavity 76 after drilling is complete and the cannula and drill bit have been withdrawn. FIG. 12I is a like FIG. 12H but from a different perspective, FIG. 12J depicts implant 58 in drilled cavity 76, and FIG. 12K depicts the same structure as FIG. 12J but from a different perspective.

### Improved Implant Delivery System Comprising Novel Directional Cannula and Combination Joint Locator and Implant Tamp

As discussed above, directional cannula 52 may be used in combination with a joint locator 50, a drill guide 54 (and drill bit 56), and an implant tamp 60 in order to deliver a fusion implant 58 into a seat formed in a sacroiliac (SI) joint 10.

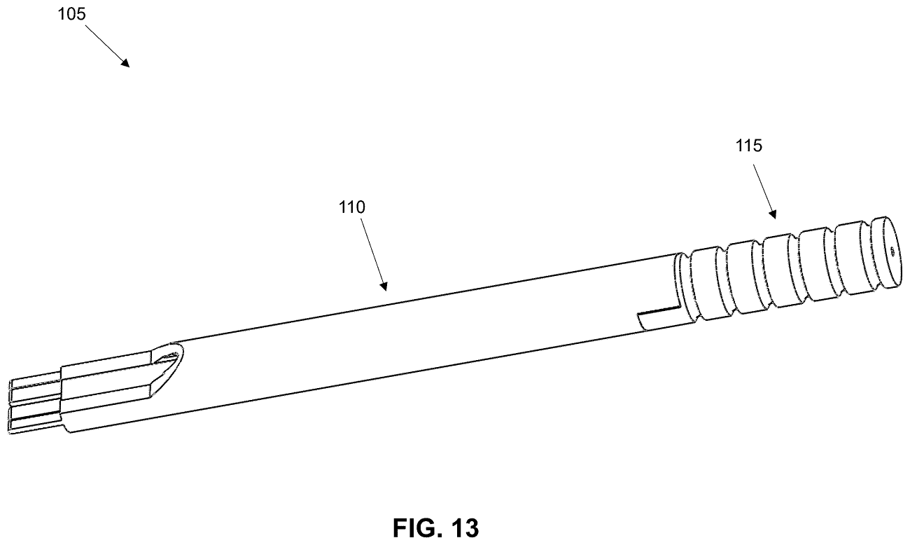

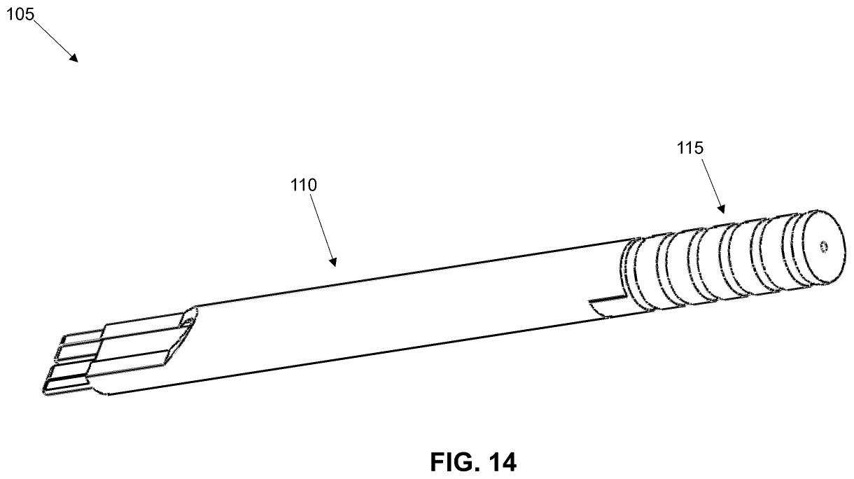

In another preferred embodiment of the present invention, and looking now at FIGS. 13 and 14, there is provided a novel implant delivery system 105 comprising a directional cannula 110 and a combined joint locator/implant tamp 115. Novel implant delivery system 105 eliminates the need for a separate joint locator and a separate implant tamp by combining the functions of a joint locator and a tamp together into combined joint locator/implant tamp 115, as will hereinafter be discussed in further detail.

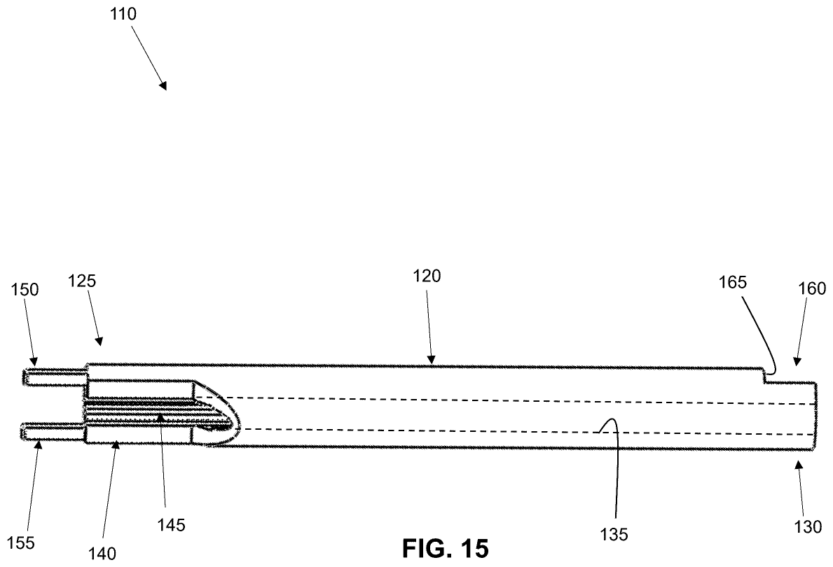

More particularly, and looking now at FIG. 15, directional cannula 110 comprises a generally-cylindrical main body 120 comprising a distal end 125, a proximal end 130, and a lumen 135 extending therebetween. Lumen 135 preferably comprises a transverse profile that matches the transverse profile of a fusion implant (e.g., the aforementioned fusion implant 58) which is to be passed down lumen 135 and into a seat formed in sacroiliac (SI) joint 10. Lumen 135 is also sized to slidably receive combined joint locator/implant tamp 115, as will hereinafter be discussed in further detail.

A portion of distal end 125 of main body 120 of directional cannula 110 is cut back so as to form a notch 140 comprising an implant lumen 145 aligned with lumen 135 such that a fusion implant (e.g., fusion implant 58) may be passed down lumen 135, along implant lumen 145 of notch 140 and into a seat formed in sacroiliac (SI) joint 10 in the same manner as the aforementioned directional cannula 52. A pair of prongs 150, 155 are mounted to (or formed integral with) distal end 125 of main body 120 and extend distally therefrom.

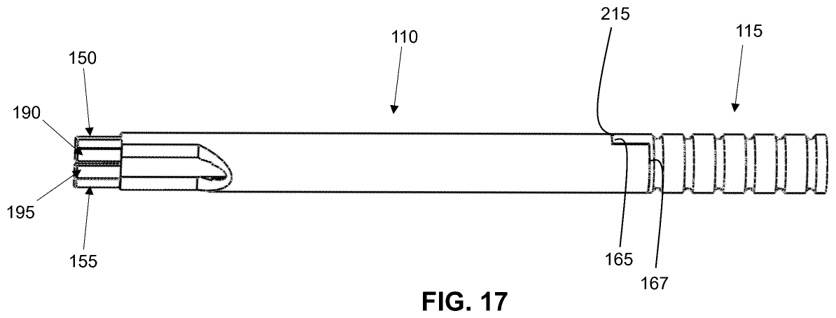

A portion of proximal end 130 of main body 120 of directional cannula 110 is cut back so as to form a proximal notch 160 defined by a distal flat 165 and a proximal end surface 167 (FIG. 17). Notch 160 and distal flat 165 permit control over the distal movement of combined joint locator/implant tamp 115 when combined joint locator/implant tamp 115 is slidably disposed in lumen 135 of main body 120 of directional cannula 110, as will hereinafter be discussed in further detail.

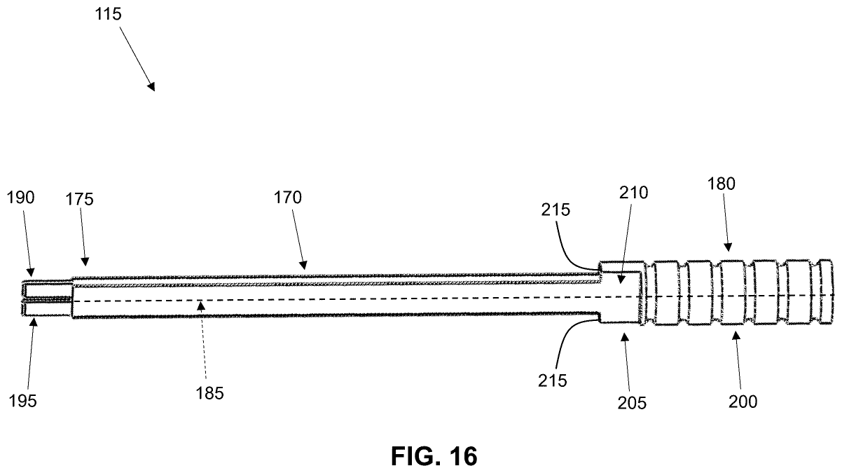

Looking now at FIG. 16, combined joint locator/implant tamp 115 generally comprises a tamp body 170 comprising a distal end 175 and a proximal end 180. Tamp body 170 of combined joint locator/implant tamp 115 may be solid or formed with a lumen 185 extending between distal end 175 and proximal end 180 of tamp body 175. Where provided, lumen 185 is preferably sized to slidably receive a guide wire therein such that combined joint locator/implant tamp 115 may be passed over a guide wire, as will be apparent to one of skill in the art in view of the present disclosure.

A pair of prongs 190, 195 are mounted to (or formed integral with) distal end 175 of tamp body 170 of combined joint locator/implant tamp 115 so as to extend distally therefrom. Proximal end 180 of tamp body 170 comprises a proximal head portion 200 (e.g., for engagement by a mallet or other tool), and a proximal stop 205 for engagement with proximal notch 160 and distal flat 165 of main body 120 of directional cannula 110, as will hereinafter be discussed in further detail.

More particularly, proximal stop 205 comprises an eccentrically-disposed radially-enlarged portion 210 having a width transverse to the longitudinal dimension of tamp body 170 which is greater than the portion of tamp body 170 extending distally from proximal stop 205. It will be appreciated that proximal stop 205 of tamp body 170 is preferably formed with a “stepped” configuration such that, as a result, proximal stop 205 defines a pair of proximal flats 215 which (i) engage with the aforementioned distal flat 165 of directional cannula 110 so as to provide a directional cannula 110 and combined joint locator/implant tamp 115 having a first length when joint locator/implant tamp 115 is disposed within lumen 135 of directional cannula 110 in a first radial position, and (i) engage with the aforementioned proximal end surface 167 of directional cannula 110 so as to provide a directional cannula 110 and combined joint locator/implant tamp 115 having a second (longer) length when joint locator/implant tamp 115 is disposed within lumen 135 of directional cannula 110 in a second radial position.

In use, and looking now at FIG. 17, when it is desired to use combined joint locator/implant tamp 115 as a joint locator, combined joint locator/implant tamp 115 is disposed in a first radial position in which proximal stop 205 of combined joint locator/implant tamp 115 is received in proximal notch 160 of proximal end 130 of main body 120 of directional cannula 110 such that proximal flats 215 engage and abut distal flat 165 of proximal notch 160. In this first radial position, it will be appreciated that combined joint locator/implant tamp 115 is fully inserted into lumen 135 of directional cannula 110 such that the distalmost tips of prongs 190, 195 of combined joint locator/implant tamp 115 extend the same distance distally as the aforementioned prongs 150, 155 of directional cannula 110, with prongs 190, 195 being received between prongs 150, 155. Thus, combined joint locator/implant tamp 115 may be inserted into lumen 135 and prongs 190, 195 of combined joint locator/implant tamp 115 may be advanced distally in order to assist in locating (and/or distracting) sacroiliac (SI) joint 10.

However, when it is desired to use combined joint locator/implant tamp 115 as an implant tamp, it is preferable to arrest distal movement of prongs 190, 195 of combined joint locator/implant tamp 115 such that prongs 190, 195 do not advance distally to the same distance as prongs 150, 155 of directional cannula 110.

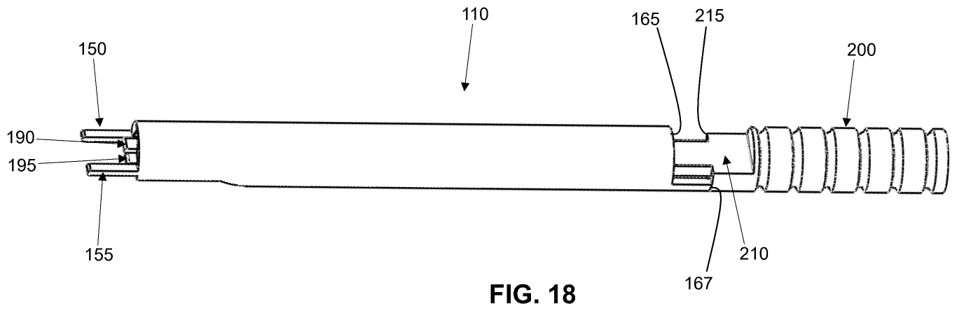

To this end, and looking now at FIG. 18, when it is desired to use combined joint locator/implant tamp 115 as an implant tamp, combined joint locator/implant tamp 115 is disposed in a second radial position (within lumen 135 of main body 120 of directional cannula 110) which is rotated 180 degrees relative to the aforementioned first radial position. When combined joint locator/implant tamp 115 is disposed in the second radial position and advanced distally into lumen 135 of directional cannula 110, proximal flats 215 of combined joint locator/implant tamp 115 engage proximal end surface 167 of proximal notch 160 of directional cannula 110. As a result of this construction, when combined joint locator/implant tamp 115 is disposed in the second radial position and thereafter advanced distally into lumen 135, prongs 190, 195 are arrested at a position proximal to the distalmost ends of prongs 150, 155 of directional cannula 110. This provides the notable advantage that the surgeon may apply a force to the proximal head portion 200 of combined joint locator/implant tamp 115 (e.g., by striking it with a mallet) with the assurance that prongs 190, 195 of combined joint locator/implant tamp 115 will not advance further distally when combined joint locator/implant tamp 115 is disposed in the second radial position. Thus a single tool (i.e., combined joint locator/implant tamp 115) may be used both as a joint locator and as an implant tamp, eliminating the need to provide these functions in separate elements.

### Simplified Method of Using Implant Delivery System Comprising Novel Directional Cannula and Combination Joint Locator and Implant Tamp

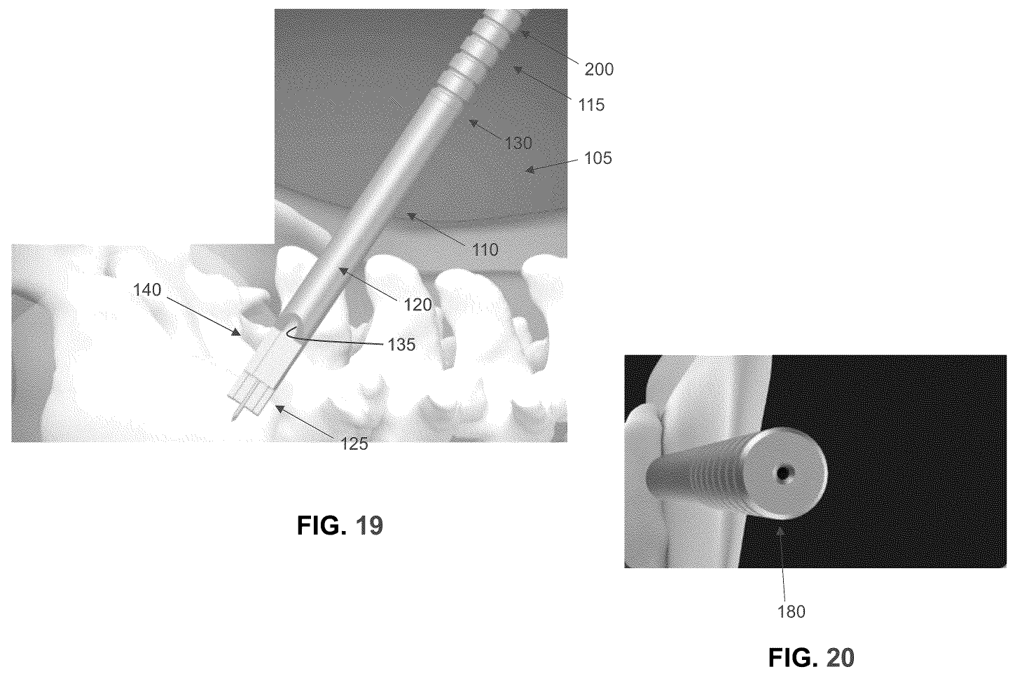

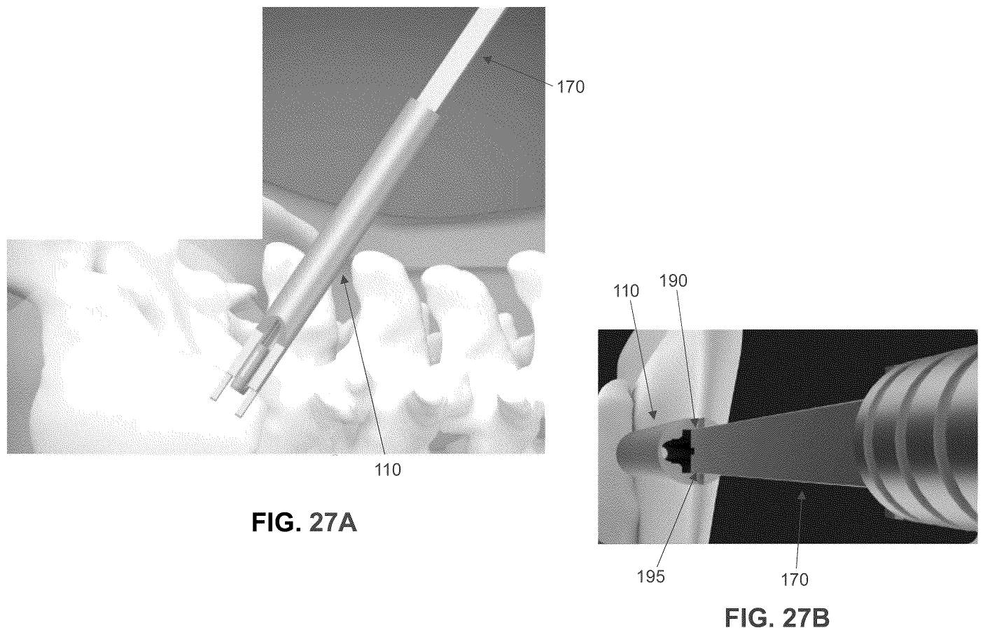

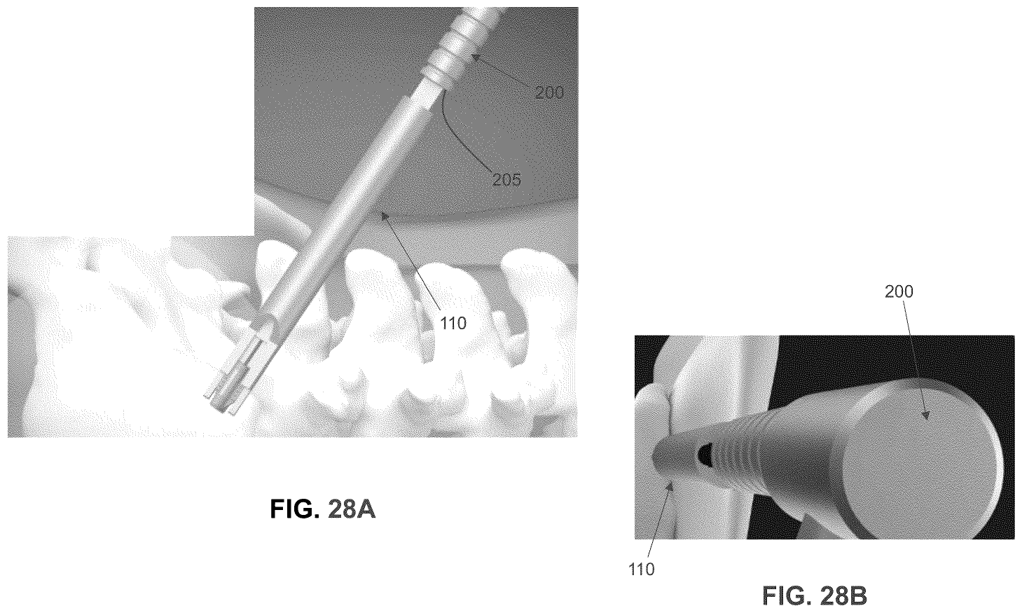

If desired, and looking now at FIGS. 19-32, directional cannula 110 and joint locator/implant tamp 115 may be used to deliver a fusion implant into the SI joint without the use of a drill.

More particularly, joint locator/implant tamp 115 is disposed in a first radial position in directional cannula 110 such that proximal stop 205 of combined joint locator/implant tamp 115 is received in proximal notch 160 of directional cannula 110 (with proximal flats 215 engaging and abutting distal flat 165 of proximal notch 160 and with the distalmost tips of prongs 190, 195 of combined joint locator/implant tamp 115 extending the same distance distally as the aforementioned prongs 150, 155 of directional cannula 110). Directional cannula 110 and joint locator/implant tamp 115 are then advanced over a guidewire (or guide pin) 687 and inserted into sacroiliac (SI) joint 10 so that the distal ends of directional cannula 110 and joint locator/implant tamp 115 are disposed near the desired location in the joint space (FIGS. 19 and 20). It will be appreciated that, if desired, guidewire (or guide pin) 687 may be omitted, and the surgeon may utilize other methods of locating the sacroiliac (SI) joint 10 so as to dispose the distal ends of directional cannula 110 and joint locator/implant tamp 115 in sacroiliac (SI) joint 10, as will be apparent to one skilled in the art.

The combined directional cannula 110 and joint locator/implant tamp 115 are then advanced distally (e.g., by using a mallet to strike proximal head portion 200 of tamp body 170) to distract SI joint 10 and/or prepare the seat in SI joint 10 to receive the fusion implant.

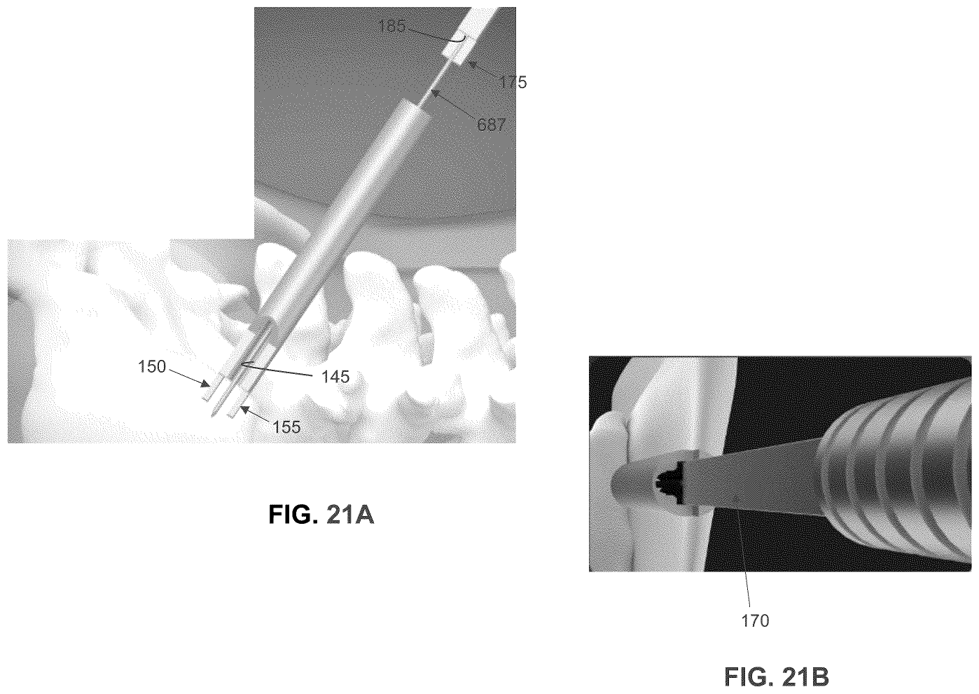

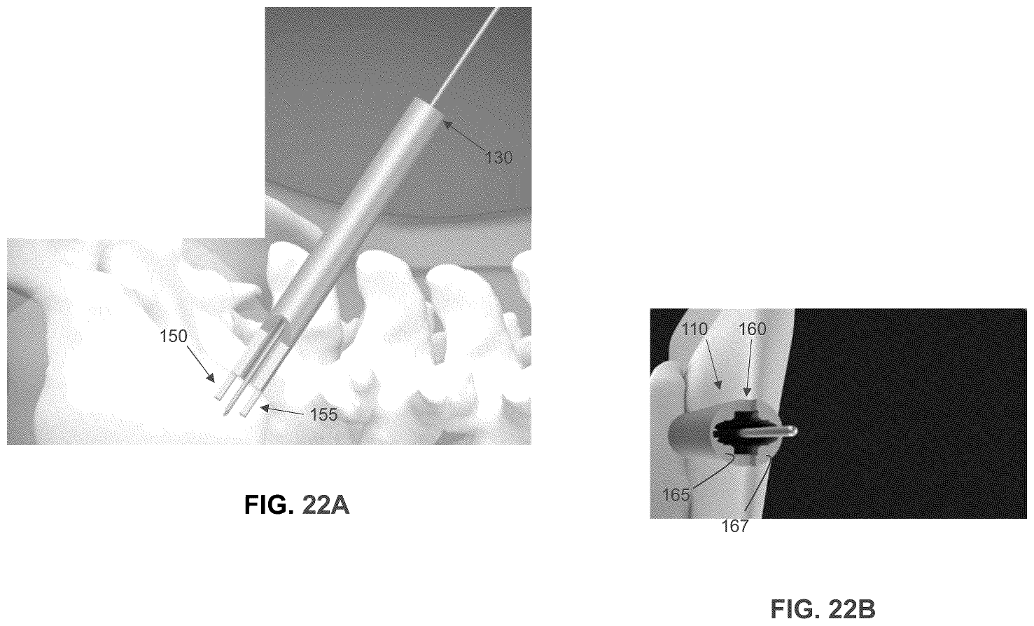

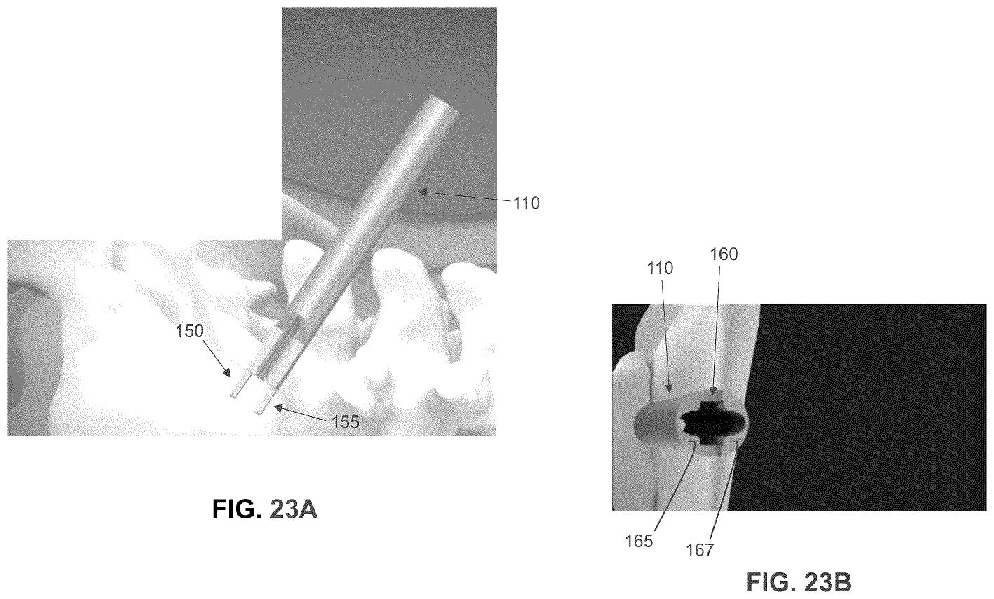

Joint locator/implant tamp 115 is then removed from lumen 135 of directional cannula 110 (FIGS. 21A and 21B), leaving directional cannula 110 disposed over guidewire (or guide pin) 687 (FIGS. 22A and 22B). Guidewire (or guide pin) 687 is then removed from the joint (FIGS. 23A and 23B).

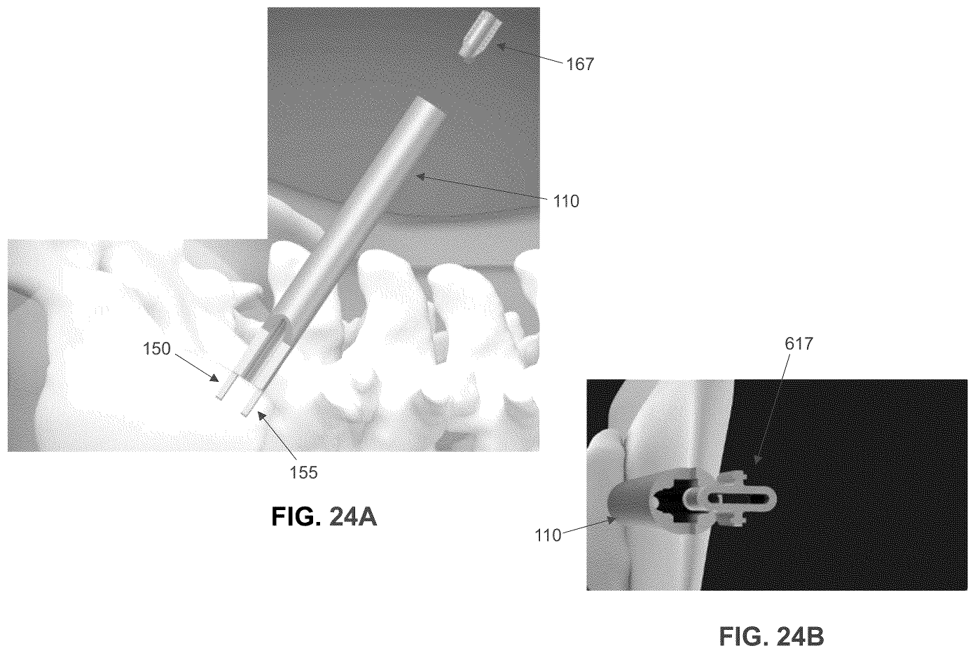

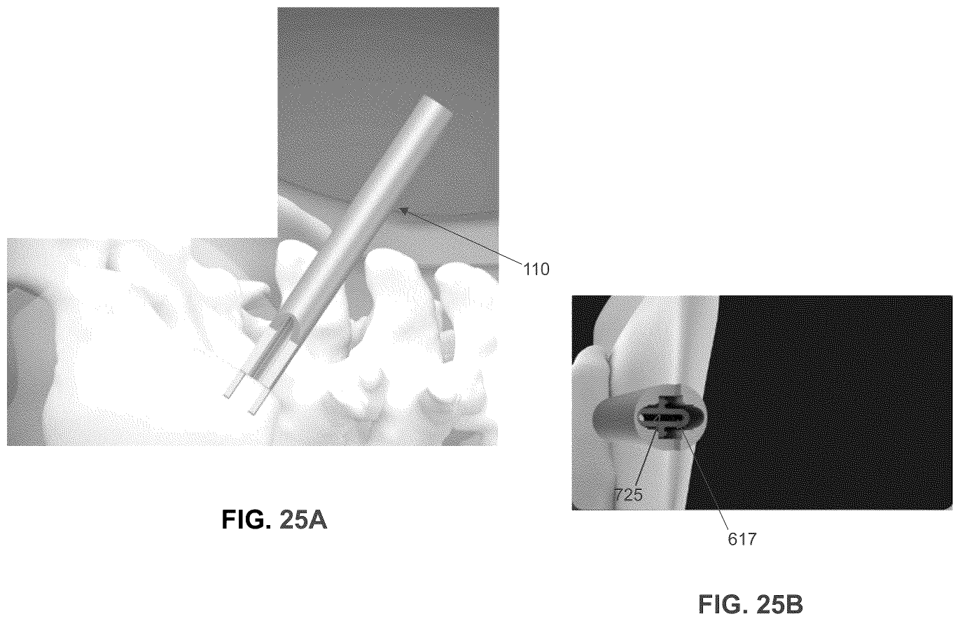

A fusion implant is inserted into lumen 135 of directional cannula 110 and passed from proximal end 130 of directional cannula 110 to distal end 125 of directional cannula 110. See FIGS. 24A, 24B, 25A and 25B.

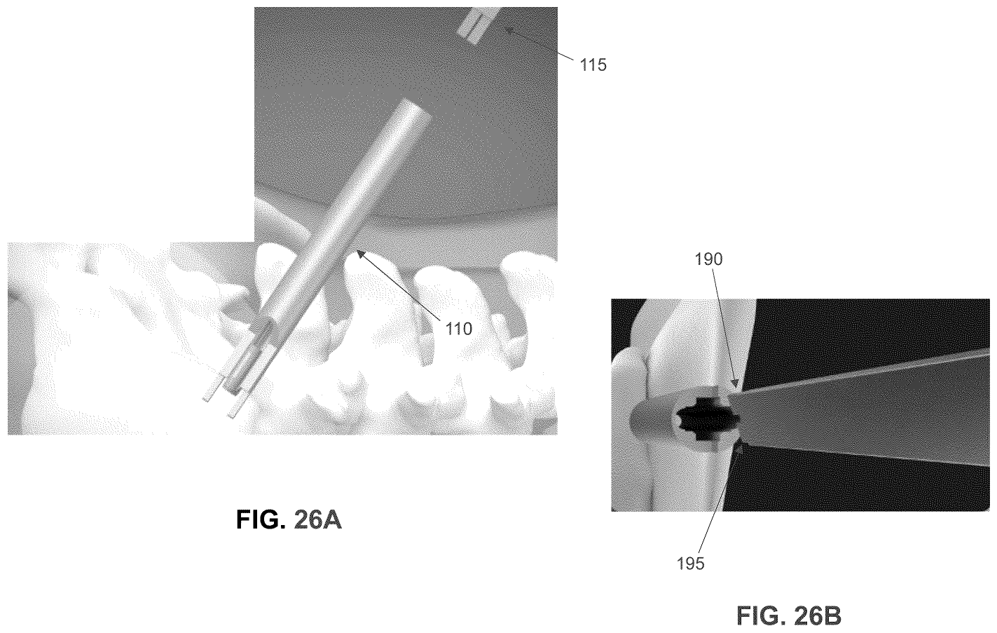

Joint locator/implant tamp 115 is then rotated 180 degrees, re-inserted into directional cannula 110 and moved distally until the distal end of tamp body 170 (e.g., the distal ends of prongs 190, 195) of joint locator/implant tamp 115 engages the proximal end of the fusion implant. See FIGS. 26A, 26B, 27A, 27B, 28A and 28B.

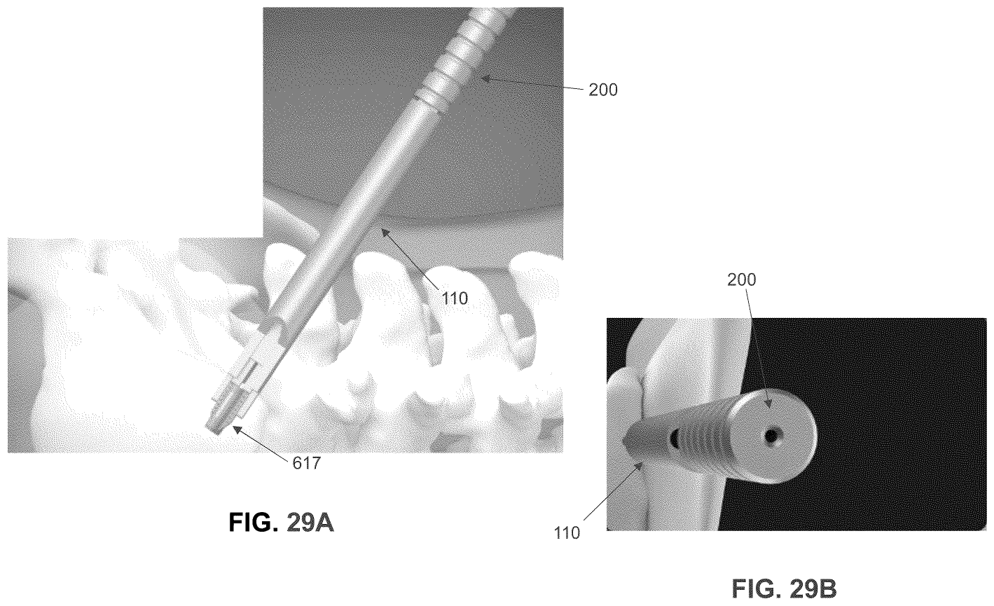

If desired, a surgeon may apply a force to the proximal head portion 200 of combined joint locator/implant tamp 115 (e.g., by striking it with a mallet) to drive the fusion implant into the joint. Since proximal flats 215 of combined joint locator/implant tamp 115 engage proximal end surface 167 of proximal notch 160 of directional cannula 110, prongs 190, 195 of combined joint locator/implant tamp 115 do not advance distally to the same distance as prongs 150, 155 of directional cannula 110 (FIGS. 29A and 29B). Thus, a surgeon can be assured that prongs 190, 195 of combined joint locator/implant tamp 115 will not advance further distally into the joint space.

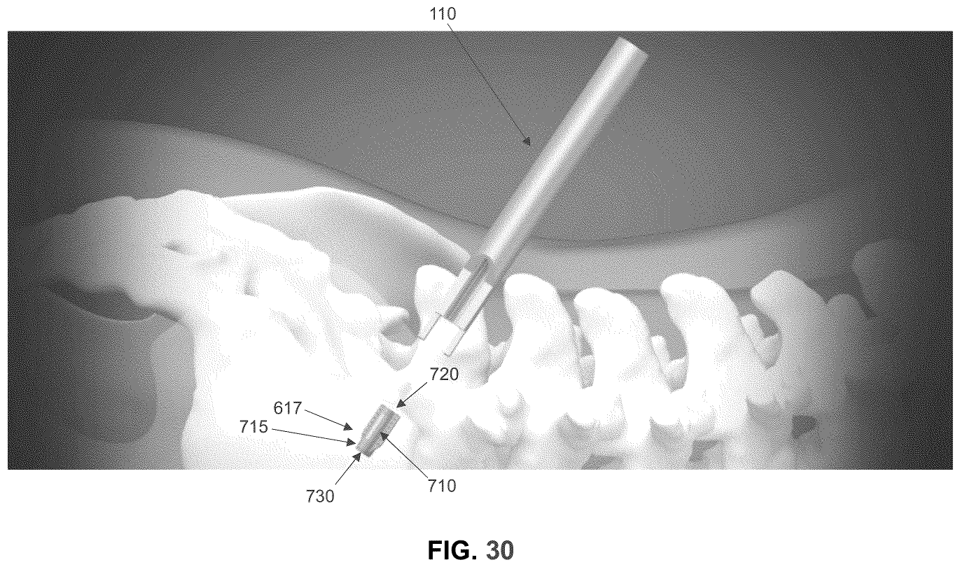

After the fusion implant has been advanced into the SI joint, joint locator/implant tamp 115 and directional cannula 110 are removed from the anatomy (i.e., by retracting joint locator/implant tamp 115 proximally), leaving the fusion implant disposed in SI joint 10. See FIG. 30.

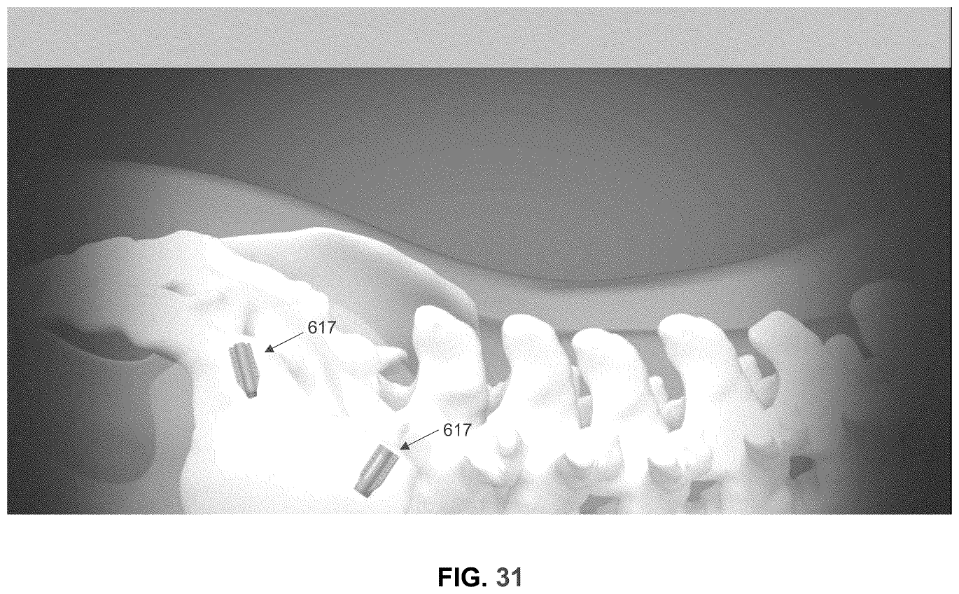

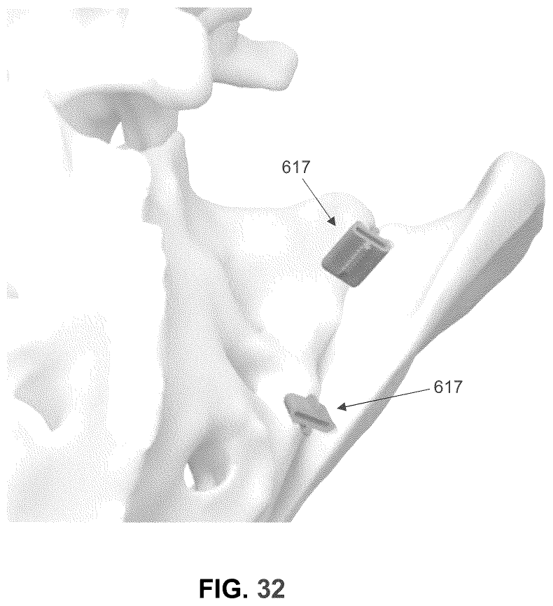

The foregoing procedure may be repeated in order to insert as many implants as desired into SI joint 10. See FIGS. 31 and 32 which show two implants inserted into the SI joint. It will be appreciated that implant delivery system 105 is suited for any manner of approach desired by the surgeon, including, but not limited to a lateral approach, an oblique approach, a posterior approach, etc.

Significantly, with the foregoing simplified procedure, there is no need to use a drill guide or drill bit to form a cavity in the bone for receiving the fusion implant.

Furthermore, if desired, the distal end of the fusion implant could be provided with a sharpened edge for driving the implant into the anatomy. To this end, and looking now at FIG. 30, a novel fusion implant 617 is provided.

Novel fusion implant 617 is generally similar to the aforementioned fusion implant 58 discussed above. Specifically, however, it should be appreciated that implant 617 (i) is formed with a sharpened (i.e., “knife edge”) distal end configured to cut into the bone of the anatomy as the implant is driven into the anatomy. Furthermore, implant 617 is formed with a hollow central lumen. If desired, implant 617 may be provided without a superior stabilizer or an inferior stabilizer.

More particularly, implant 617 generally comprises a body 710 comprising a distal end 715, a proximal end 720, and a cavity 725 formed therebetween. In a preferred form of the invention, cavity 725 opens on proximal end 720 (see FIG. 25B). Distal end 715 of body 710 preferably comprises a sharpened distal leading edge 730 configured to cut into bone as implant 617 is driven distally into the anatomy.

Although the method of FIGS. 19-32 is shown with the use of a guidewire (or guide pin) 687, it is important to note that the guide wire (or guide pin) 687 is optional. If desired, the foregoing procedure can be further simplified by inserting directional cannula 110 and joint locator/implant tamp 115 into the joint without the use of a guidewire (or guide pin).

Furthermore, although the foregoing method is described in the context of using directional cannula 110 and joint locator/implant tamp 115 to deliver fusion implant 58 or fusion implant 617 to the SI joint, it is important to note that directional cannula 110 and joint locator/implant tamp 115 can be used to implant any fusion implant, whether it is a fusion implant having a main body (either hollow or solid) with one or more stabilizers or a fusion implant having a main body (either hollow or solid) without stabilizers.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for positioning a fusion implant into a sacroiliac joint, the apparatus comprising:
a directional cannula comprising:
a main body having a distal end, a proximal end and a lumen extending between the distal end and the proximal end;
two cannula prongs extending distally from the distal end of the main body;
a distal cut-out formed in the distal end of the main body; and
a proximal notch formed in the proximal end of the main body so as to define a first proximal end surface and a second proximal end surface, wherein the first proximal end surface is disposed distal to the second proximal end surface; and
a combined implant tamp and joint locator configured to be disposed in the lumen of the directional cannula, the combined implant tamp and joint locator comprising:
a tamp body having a distal end and a proximal end;
two tamp prongs extending distally from the distal end of the tamp body; and
a proximal stop formed at the proximal end of the tamp body so as to define a first stop surface and a second stop surface, wherein the first stop surface is disposed distal to the second stop surface.

2. The apparatus of claim 1 wherein the first stop surface of the combined implant tamp and joint locator is configured to engage with the first proximal end surface of the directional cannula when the combined implant tamp and joint locator is disposed in the lumen of the main body in a first radial position, and the second stop surface of the combined implant tamp and joint locator is configured to engage with the second proximal end surface of the directional cannula when the combined implant tamp and joint locator is disposed in the lumen of the main body in a second radial position.

3. The apparatus of claim 2 wherein when the combined implant tamp and joint locator is disposed in the first radial position, a distalmost end of each of the two cannula prongs of the directional cannula are aligned with a distalmost end of each of the two tamp prongs of the combined implant tamp and joint locator, and further wherein when the combined implant tamp and joint locator is disposed in the second radial position, the distalmost end of each of the two cannula prongs of the directional cannula extend distal to the distalmost end of each of the two tamp prongs of the combined implant tamp and joint locator.

4. The apparatus of claim 2 wherein the second radial position is disposed 180 degrees relative to the first radial position.

5. The apparatus of claim 1 wherein the proximal end of the tamp body comprises an enlarged proximal head portion.

6. The apparatus of claim 1 wherein the lumen of the main body of the directional cannula comprises a transverse profile that matches a transverse profile of the fusion implant.

7. The apparatus of claim 1 wherein the lumen of the directional cannula comprises a transverse profile characterized by a first lateral dimension and a second lateral dimension disposed perpendicular to the first lateral dimension, wherein the first lateral dimension is larger than the second lateral dimension.

8. The apparatus of claim 1 wherein the tamp body comprises a guidewire lumen extending from the distal end of the tamp body to the proximal end of the tamp body.

9. The apparatus of claim 1 wherein the distal cut-out of the main body of the directional cannula comprises a distal end surface comprising a generally planar surface defined by a plane transverse to the longitudinal axis of the lumen, the generally planar surface being configured to seat securely against the sacrum;
an intermediate surface extending at an angle to the to the longitudinal axis of the lumen, wherein the intermediate surface has a length that is less than half of a diameter of the main body of the directional cannula, the intermediate surface being configured to seat securely against the ilium; and
a pair of side surfaces extending parallel to the longitudinal axis of the lumen from the distal end surface to the intermediate surface.

10. The apparatus of claim 1 wherein the fusion implant comprises an implant body, a first stabilizer extending outwardly from a top surface of the implant body and a second stabilizer extending outwardly from a bottom surface of the implant body.

11. The apparatus of claim 10 wherein the first stabilizer and the second stabilizer are diametrically opposed from one another.

12. The apparatus of claim 1 wherein the fusion implant comprises an implant body having a distal end, a proximal end and a lumen extending between the distal end and the proximal end.

13. The apparatus of claim 12 wherein the distal end of the implant body comprises a cutting edge.

14. A method of positioning a fusion implant into a sacroiliac joint, the method comprising:
inserting a combined implant tamp and joint locator into a lumen of a directional cannula;
inserting the directional cannula and the combined implant tamp and joint locator into the sacroiliac joint to prepare a cavity in the sacroiliac joint for receiving the fusion implant;
withdrawing the combined implant tamp and joint locator from the lumen of the directional cannula;
inserting the fusion implant into the lumen of the directional cannula;
re-inserting the combined implant tamp and joint locator into the lumen of the directional cannula; and
using the combined implant tamp and joint locator to tamp the fusion implant into the cavity in the sacroiliac joint.

15. The method of claim 14 wherein the directional cannula comprises:
a main body having a distal end, a proximal end and a lumen extending between the distal end and the proximal end;
two cannula prongs extending distally from the distal end of the main body;
a distal cut-out formed in the distal end of the main body; and
a proximal notch formed in the proximal end of the main body so as to define a first proximal end surface and a second proximal end surface, wherein the first proximal end surface is disposed distal to the second proximal end surface; and
wherein the combined implant tamp and joint locator comprises:
a tamp body having a distal end and a proximal end;
two tamp prongs extending distally from the distal end of the tamp body; and
a proximal stop formed at the proximal end of the tamp body so as to define a first stop surface and a second stop surface, wherein the first stop surface is disposed distal to the second stop surface.

16. The method of claim 15 wherein when the combined implant tamp and joint locator is disposed in the lumen of the main body in a first radial position, the first stop surface of the combined implant tamp and joint locator is configured to engage with the first proximal end surface of the directional cannula, and when the combined implant tamp and joint locator is disposed in the lumen of the main body in a second radial position, the second stop surface of the combined implant tamp and joint locator is configured to engage with the second proximal end surface of the directional cannula.

17. The method of claim 16 wherein when the combined implant tamp and joint locator is disposed in the first radial position, a distalmost end of each of the two cannula prongs of the directional cannula are aligned with a distalmost end of each of the two tamp prongs of the combined implant tamp and joint locator, and further wherein when the combined implant tamp and joint locator is disposed in the second radial position, the distalmost end of each of the two cannula prongs of the directional cannula extend distal to the distalmost end of each of the two tamp prongs of the combined implant tamp and joint locator.

18. The apparatus of claim 16 wherein the second radial position is disposed 180 degrees relative to the first radial position.

19. The method of claim 14 further comprising applying a force to the combined implant tamp and joint locator so as to advance the combined implant tamp and joint locator through the lumen of the directional cannula, whereby to seat the fusion implant in the sacroiliac joint.

20. The method of claim 14 wherein the proximal end of the combined implant tamp and joint locator comprises an enlarged proximal head portion.

21. The method of claim 14 wherein prior to re-inserting the combined implant tamp and joint locator into the lumen of the directional cannula, the combined implant tamp and joint locator is rotated 180 degrees.

22. The method of claim 15 wherein when the directional cannula and the combined implant tamp and joint locator are disposed in the sacroiliac joint, the two cannula prongs and the two tamp prongs engage the sacroiliac joint, with a first portion of the cut-out of the main body of the directional cannula seated against the sacrum and a second portion of the cut-out of the main body of the directional cannula seated against the ilium.

23. The method of claim 14 wherein a distal end of the implant body comprises a cutting edge.

24. The method of claim 14 further comprising a guide, wherein inserting the directional cannula and the combined implant tamp and joint locator into the sacroiliac joint comprises passing the directional cannula and the combined implant tamp and joint locator over the guide and into the sacroiliac joint.

25. The method according to claim 24 wherein the guide comprises one from the group consisting of a guide wire and a guide pin.

26. A method of positioning a fusion implant into a sacroiliac joint, the method comprising:
providing a directional cannula comprising:
a main body having a distal end, a proximal end and a lumen extending between the distal end and the proximal end;
two cannula prongs extending distally from the distal end of the main body;
a distal cut-out formed in the distal end of the main body; and
a proximal notch formed in the proximal end of the main body so as to define a first proximal end surface and a second proximal end surface, wherein the first proximal end surface is disposed distal to the second proximal end surface;
providing a combined implant tamp/joint locator comprising:
a tamp body having a distal end and a proximal end;
two tamp prongs extending distally from the distal end of the tamp body; and
a proximal stop formed at the proximal end of the tamp body so as to define a first stop surface and a second stop surface, wherein the first stop surface is disposed distal to the second stop surface;
inserting the combined implant tamp and joint locator into the lumen of the directional cannula in a first radial position such that the first stop surface of the combined implant tamp and joint locator engages the first proximal end surface of the directional cannula and distalmost ends of the two cannula prongs of the directional cannula are aligned with distalmost ends of the two tamp prongs of the combined implant tamp and joint locator;
inserting the directional cannula and the combined implant tamp and joint locator into the sacroiliac joint such that the two cannula prongs and the two tamp prongs engage the sacroiliac joint;
withdrawing the combined implant tamp and joint locator from the lumen of the directional cannula;
inserting the fusion implant into the lumen of the directional cannula;
rotating the combined implant tamp and joint locator 180 degrees to a second radial position;
inserting the rotated combined implant tamp and joint locator into the lumen of the directional cannula in the second radial position;
applying a force to the proximal end of the combined implant tamp and joint locator to seat the fusion implant in the sacroiliac joint, wherein the second stop surface of the combined implant tamp and joint locator and the second proximal end surface of the directional cannula prevent the two tamp prongs of the combined implant tamp and joint locator from extending distal to the two cannula prongs of the directional cannula; and
removing the combined implant tamp and joint locator and directional cannula from the sacroiliac joint.
